(19) United States
(12) Patent Application Publication
(10) Pub. No.: US 2011/0287089 A1
(43) Pub. Date: Nov. 24, 2011

(54) USE OF A SACCHAROMYCES CEREVISIAE MITOCHONDRIAL NUCLEIC ACIDS FRACTION FOR IMMUNE STIMULATION

(76) Inventor: Karola Rittner, Strasbourg (FR)

(21) Appl. No.: 13/144,280

(22) PCT Filed: Jan. 8, 2010
(86) PCT No.: PCT/EP2010/050150

§ 371 Date: Aug. 2, 2011

Related U.S. Application Data
(60) Provisional application No. 61/202,515, filed on Mar. 6, 2009.

(30) Foreign Application Priority Data
Jan. 13, 2009 (EP) 09305032.6

(51) Int. Cl. A61K 9/127; A61K 39/12; A61P 37/04; A61P 37/08; A61P 31/00; A61K 39/00; A61P 35/00

(52) U.S. Cl. 424/450; 424/274.1; 424/204.1; 424/233.1; 424/207.1; 424/229.1; 424/218.1; 424/232.1

(57) ABSTRACT

The present invention relates to the use of a Saccharomyces cerevisiae mitochondrial nucleic acids fraction and an antigen for the preparation of a pharmaceutical composition intended to orient the immune response toward a Th1 type response directed against said antigen, more particularly for the prevention and/or the treatment of cancer, infectious disease and allergy. Adjuvant compositions with synergic effect, vaccine compositions with synergic effect, and kits of part are also provided. Methods of treatment of individuals thereof are also provided.

### TECHNICAL FIELD

The present invention pertains generally to adjuvants. In particular, the invention relates to the use of a Saccharomyces cerevisiae mitochondrial nucleic acids fraction with adjuvant effect for the preparation of pharmaceutical compositions intended to orient the immune response toward a Th1 type response directed against specific antigens.

### BACKGROUND OF THE INVENTION

For years, vaccination techniques have essentially consisted in the introduction into an animal of an antigen (e.g. a protein, a killed or attenuated virus) in order to raise an immune response directed against an infectious organism. Since the end of the 80's, new vaccination techniques have appeared which consist in the introduction into an animal of a vector comprising a nucleic acid sequence coding for the antigen. For example, a live vaccinia virus encoding a rabies glycoprotein has been successfully used for the elimination of terrestrial rabies in Western European countries (CLIQUET, et al. Elimination of terrestrial rabies in Western European countries. Developments in biologicals, 2004, vol. 119, p. 185-204). The major advantage of nucleic acid immunization is that both cellular (including CD4+ and CD8+ T cells) and humoral immune responses can be induced because the encoded antigen is processed through both endogenous and exogenous pathways, and peptide epitopes are presented by major histocompatibility complexes (MHC) class I as well as class II complexes (HAUPT, et al. The Potential of DNA Vaccination against Tumor-Associated Antigens for Antitumor Therapy. Experimental Biology and Medicine. 2002, vol. 227, p. 227-237).

The efficient generation of a Cytotoxic T Lymphocyte (CTL) response has paved the way for the prophylactic or therapeutic treatment of cancer by nucleic acid vaccination. Many tumor cells express specific antigen(s) called TAA (for tumor associated antigen), but these antigens are poorly recognized by the immune system which is down regulated by factors at the periphery of tumor. The vaccination of patients with a nucleic acid encoding a TAA leads to the expression of the TAA in an environment where the immune system is fully effective and generates an immune response specifically directed against the tumor cells.

However, while vaccination continues to be the most successful interventionist health policy to date, infectious disease and cancer remain a significant cause of death worldwide. A primary reason that vaccination is not able to generate effective immunity is a lack of appropriate adjuvants capable of initiating the desired immune response. Moreover, most conventional adjuvants are poorly defined, complex substances that fail to meet the stringent criteria for safety and efficacy desired in new generation vaccines.

A new generation of adjuvants that work by activating innate immunity presents exciting opportunities to develop safer, more potent vaccines. The family of Toll-like receptors (TLRs) appears to play a pivotal role in the innate immune system for the detection of highly conserved, pathogen-expressed molecules. To enable the rapid detection of infection, each of the 10 TLRs currently known to be expressed in humans has apparently evolved to be stimulated in the presence of certain types of pathogen-expressed molecules, which are either not expressed in host cells, or are sequestered in cellular compartments where they are unavailable to the TLRs. Activation of a TLR by an appropriate pathogen molecule acts as an “alarm signal” for initiation of the appropriate immune defenses. These TLR activators have also been successfully used alone to boost the natural immune response raised against pathogens or tumoral cells. For example, CpG oligodeoxynucleotides (ODNs) are TLR9 agonists that show promising results as vaccine adjuvants and in the treatment of cancers, infections, asthma and allergy. One of them. CPG-7909, was developed for the treatment of cancer as monotherapy and as an adjuvant in combination with chemo- and immunotherapy. Phase I and II trials have tested this drug in several hematopoietic and solid tumors (MURAD, et al. CPG-7909 (PF-3512676, ProMune): toll-like receptor-9 agonist in cancer therapy. Expert opinion on biological therapy. 2007, vol. 7, no. 8, p. 1257-66).

The nature of an immune response reflects the profile of antigen-specific lymphocytes that are stimulated by the immunization. Lymphocytes, particularly T cells, consist of subpopulations that may be stimulated by different types of antigens and perform different effector functions. For instance, in viral infections viral antigens are synthesized in infected cells and presented in association with class I MHC molecules, leading to the stimulation of CD8⁺ class I MHC-restricted CTLs. In contrast, extracellular microbial antigens are endocyted by APCs, processed, and presented preferentially in association with class II MHC molecules. This activates CD4⁺, class II MHC-restricted helper T cells, leading to antibody production and macrophage activation but relatively inefficient development of CTLs. Even within the population of CD4⁺ helper T cells there are subsets that produce distinct cytokines in response to antigenic stimulation. Naive CD4⁺ T cells produce mainly the T cell growth factor, interleukin 2 (IL-2), upon initial encounter with antigen. Antigenic stimulation may lead to the differentiation of these cells, sometimes into a population called Th0, which produce cytokines, and subsequently into subsets called Th1 and Th2, which have relatively restricted profiles on cytokine production and effector functions. Th1 cells secrete gamma interferon (IFN-γ), interleukin-2 (IL-2), which activates macrophages, and are the principal effectors of cell-mediated immunity against intracellular microbes and of delayed type hypersensitivity reactions. The antibody isotypes stimulated by Th1 cells are effective at activating complement and opsonizing antigens for phagocytosis. Therefore, the Th1 cells trigger phagocyte-mediated host defense. Infections with intracellular microbes tend to induce the differentiation of naive T cells into Th1 subset, which promotes phagocytic elimination of the microbes. Th2 cells, on the other hand, produce interleukin-4 (IL-4) which stimulates IgE antibody production, interleukin-5 (IL-5) which is an eosinophil-activating factor and interleukin-10 (IL-10) and interleukin-13 (IL-13) which together with interleukin-4 (IL-4) suppress cell-mediated immunity. Therefore, the Th2 cells is mainly responsible for phagocyte-independent host defense, e.g. against certain helminthic parasites, which is mediated by IgE and eosinophils, and for allergic reactions, which are due to IgE-dependent-activation of mast cells and basophils (ABBAS A. K. and al., Cellular and molecular Immunology, W. B. Saunders Co.)

Winkler et al. (WINKLER, S., M. Willheim, K. Baler, et al. 1998. Reciprocal regulation of Th1- and Th2-cytokine-producing T cells during clearance of parasitemia in Plasmodium falciparum malaria, Infect. Immun. 66:6040-6044.) have shown in patients with uncomplicated P. falciparum malaria the role of IFN-γ as a key molecule in human antimalarial host defense, and they do not support a direct involvement of interleukin-4 (IL-4) in the clearance of P. falciparum parasites. Moreover, it has been shown that, for the same given antigen, it is the adjuvant which orients toward the predominant isotype during the antibody response (TOELLNER K.-M. et al. J. Exp. Med. 1998, 187: 1193). For instance, it is known that aluminium salts, such as Alhydrogel, induce, in mice, an essentially Th2 type response and promote the formation of IgG1 or even of IgE (ALLISON A. C. In Vaccine design—The role of cytokine networks Vol. 293, 1-9 Plenum Press 1997), which can pose problems in subjects with an allergic predisposition.

With this regard there is currently still a need to have available adjuvants capable of orienting the immune response toward a Th1 type response against antigens.

### DISCLOSURE OF THE INVENTION

The applicant has surprisingly found that a specific Saccharomyces cerevisiae mitochondrial nucleic acids fraction is TLRs activator and is capable to orient the immune response toward a Th1 type response against antigens.

As used throughout the entire application, a “Th1 type response” refers to one which stimulates the production gamma interferon (IFN-γ), interleukin-2 (IL-2) and/or interleukin-12 (IL-12).

As used throughout the entire application, “a” and “an” are used in the sense that they mean “at least one”, “at least a first”, “one or more” or “a plurality” of the referenced components or steps, unless the context clearly dictates otherwise.

As used throughout the entire application, “and/or” wherever used herein includes the meaning of “and”, “or” and “all or any other combination of the elements connected by said term”.

As used throughout the entire application, “comprising” and “comprise” are intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The present invention relates to the use of a Saccharomyces cerevisiae mitochondrial nucleic acids fraction and an antigen for the preparation of a pharmaceutical composition intended to orient the immune response toward a Th1 type response directed against said antigen, characterized in that said Saccharomyces cerevisiae mitochondrial nucleic acids fraction is prepared by a method comprising the following steps:
a) culture of Saccharomyces cerevisiae in a culture medium allowing their growth followed by centrifugation of said culture;
b) grinding of the Saccharomyces cerevisiae pellet obtained in step a);
c) centrifugation of the mixture obtained in step b);
d) ultracentrifugation of the supernatant obtained in step c);
e) extraction of nucleic acids from the pellet obtained in step d);
f) recovering of the nucleic acids fraction from the supernatant obtained in step e).

Saccharomyces cerevisiae (S.c.) is well described (Meyen ex E. C. Hansen, 1883) and is commercially available (e.g. S.c. DSM No. 1333 ATCC 9763; S.c. DSM NO 70464 NCYC 1414; S.c. DSM NO 2155 ATCC 7754; S.c. DSM No. 70869; S.c. DSM No. 70461 NCYC 1412; S.c. AH109 Clontech; S.c. Y187 Clontech; S.c. W303 Biochem). In a preferred embodiment of the invention, the Saccharomyces cerevisiae used is Saccharomyces cerevisiae AH109 (Clontech) as described in Example 1. In another preferred embodiment of the invention, the Saccharomyces cerevisiae used is Saccharomyces cerevisiae W303 (Biochem) as described in Example 1.

Methods for culturing Saccharomyces cerevisiae in step a) are well known to the one skilled in the art (Guthrie, C. & Fink, G. R. (1991) Guide to yeast genetics and molecular biology—Methods in Enzymology (Academic Press, San Diego, Calif.) 194:1-932 Heslot, H. & Gaillardin, C., eds. (1992) Molecular Biology and Genetic Engineering of Yeasts, CRC Press, Inc.). Culture media allowing the growth of Saccharomyces cerevisiae are well described (e.g. Medium 1017 YPG medium DSMZ; Medium 186 YM medium DSMZ; Medium 393 YPD medium DSMZ) and some are commercially available (e.g. YPD medium Clontech). Culture media allowing the growth of Saccharomyces cerevisiae comprise at least yeast extract, peptone and glucose. Culture media used may be supplemented with one or more nutrients such as for instance amino acids, vitamins, salts and/or miscellaneous. Some of them are commercially available (e.g. YPDA medium Clontech corresponding to YPD medium supplemented with adenine). The culture conditions such as for instance nutrients, temperature and duration are well known to those ordinary skilled in the art (Guthrie, C. & Fink, G. R. (1991) Guide to yeast genetics and molecular biology—Methods in Enzymology (Academic Press, San Diego, Calif.) 194:1-932 Heslot, H. & Gaillardin, C., eds. (1992) Molecular Biology and Genetic Engineering of Yeasts, CRC Press, Inc.). In a preferred embodiment of the invention, method and conditions as described in Example 1 are used, wherein Saccharomyces cerevisiae AH109 or W303 is cultured in a culture medium comprising yeast extract (1%), peptone (1%) and glucose (2%) supplemented with adenine (100 μg/ml) at a temperature between 28° C. and 30° C.

Step a) of centrifugation of the Saccharomyces cerevisiae culture previously obtained is performed under an acceleration and during a time suitable to pellet all the Saccharomyces cerevisiae. The person skilled in the art is able to determine which speed and which duration are the most appropriate. Step a) of centrifugation of the Saccharomyces cerevisiae culture previously obtained is preferably performed under an acceleration of 3500 rpm during at least 15 minutes as described in Example 1.

Step b) of grinding of the Saccharomyces cerevisiae pellet obtained in step a) may be carried out by methods, means and any system or apparatus well known to a person skilled in the art (e.g. RIEDER S E, Emr S D, Overview of subcellular fractionation procedures for the yeast Saccharomyces cerevisiae, Curr Protoc Cell Biol. 2001 May; Chapter 3:Unit 3.7.; RIEDER S E, Emr S D, Isolation of subcellular fractions from the yeast Saccharomyces cerevisiae, Curr Protoc Cell Biol. 2001 May; Chapter 3:Unit 3.8.; HARJU S, Fedosyuk H. Peterson K R., Rapid isolation of yeast genomic DNA: Bust n' Grab, BMC Blotechnol 2004 Apr. 21; 4:8.), such as manual grinding using a mortar and pestle; grinding using a vortex (e.g. desktop vortex Top Mix 94323 Bioblock Scientifique) in the presence of glass beads having preferably a diameter between 0.1 and 5 mm and more preferably a diameter of 0.7 mm; grinding using a vortex mixer (commercially available from e.g. Labnet); grinding by liquid-based homogenization using a Dounce homogenizer (commercially available from e.g. Kontes), using a Potter-Elvehjem homogenizer (commercially available from e.g. Kontes) or using a SLM Aminco French press; mechanical grinding using a Waring Blender Polytron (commercially available from e.g. Brinkmann Instruments); grinding by sonication using a Sonicator (commercially available from e.g. Biologics; Misonix; GlenMills); or grinding by freeze/thaw. Step b) of grinding of the Saccharomyces cerevisiae pellet obtained in step a) is preferably performed at a temperature of 4° C. According to notably the initial quantity of the Saccharomyces cerevisiae pellet obtained in step a) to be treated, the person skilled in the art is able to determine which one of the grinding method previously described is the most appropriate. The person skilled in the art is moreover able to determine the grinding conditions in step b) such as for instance speed and duration. In a preferred embodiment of the invention, step b) of grinding of the Saccharomyces cerevisiae pellet obtained in step a) is performed by grinding using a vortex in the presence of glass beads. The glass beads have preferably a diameter between 0.1 and 5 mm and more preferably a diameter of 0.7 mm. The grinding is preferably performed on a base of 1 to 20 cycles, more preferably 5 cycles, of a duration of 30 seconds to 2 minutes per cycle, more preferably 1 minute per cycle. In a more preferred embodiment of the invention, step b) of grinding of the Saccharomyces cerevisiae pellet obtained in step a) is performed by grinding using a vortex in the presence of glass beads, wherein the glass have a diameter of 0.7 mm and wherein the grinding is performed on a base of 5 cycles of a duration of 1 minute per cycle as described in Example 1.

The grinding of the Saccharomyces cerevisiae pellet obtained in step a) may be preceded by a digestion in the presence of protease enzymes. Protease enzymes preferably used according to the present invention are β-glycanases from yeast cell wall such as for instance (endo or exo)β-1,3-glycanase or (endo or exo)β-1,4-glycanase, including but not limited to zymolyase and oxalyticase. According to the present invention, reactions conditions, pH of solution, temperature and duration of reaction are preferably adjusted to the optimum conditions for the activity of the protease enzyme(s) chosen. The person skilled in the art is able to determine these conditions (RIEDER S E, Emr S D, Overview of subcellular fractionation procedures for the yeast Saccharomyces cerevisiae, Curr Protoc Cell Biol. 2001 May; Chapter 3:Unit 3.7.; RIEDER S E, Emr S D, Isolation of subcellular fractions from the yeast Saccharomyces cerevisiae, Curr Protoc Cell Biol. 2001 May; Chapter 3:Unit 3.8). In another preferred embodiment of the invention, step b) of grinding of the Saccharomyces cerevisiae pellet obtained in step a) is therefore preceded by a digestion of the Saccharomyces cerevisiae pellet obtained in step a) in the presence of one or more protease enzymes, preferably zymolyase or oxalyticase or combination thereof.

Step c) of centrifugation of the mixture obtained in step b) is performed under an acceleration and during a time suitable to pellet the membrane debris as well as the nuclei. The person skilled in the art is able to determine which speed and which duration are the most appropriate. Step c) of centrifugation of the mixture obtained in step b) is preferably performed under an acceleration of 4000 rpm during 10 minutes as described in Example 1. Step c) of centrifugation of the mixture obtained in step b) is preferably performed is preferably performed at a temperature of 4° C.

Step d) of ultracentrifugation of the supernatant obtained in step c) is performed under an acceleration and during a time suitable to pellet the mitochondria. The person skilled in the art is able to determine which speed and which duration are the most appropriate. Step d) of ultracentrifugation of the supernatant obtained in step c) is preferably performed under an acceleration of 39000 rpm during 90 minutes as described in Example 1. Step d) of ultracentrifugation of the supernatant obtained in step c) is preferably performed at a temperature of 4° C.

Methods for extraction of nucleic acids are well known to the one skilled in the art. Step e) of extraction of nucleic acids from the pellet comprising the mitochondria obtained in step d) may be for instance performed by phenol-dichloromethane extraction or phenol-chloroform extraction (e.g. CHOMCZYNSKI P. and Sacchi N. (1987), “Single-step method of RNA isolation by acid guanidinium thiocyanate-phenol-chloroform extraction” Anal. Biochem, 162: 156-159). In a preferred embodiment of the invention, method and conditions as described in Example 1 are used, wherein step e) of extraction of nucleic acids from the pellet comprising the mitochondria obtained in step d) is preferably performed by phenol-dichloromethane extraction.

Step f) of recovering of the nucleic acids fraction from the supernatant obtained in step e) is performed by alcohol precipitation well known to the one skilled in the art (e.g. HARJU S. Fedosyuk H. Peterson K R., Rapid isolation of yeast genomic DNA: Bust n' Grab, BMC Biotechnol. 2004 Apr. 21; 4:8). In a preferred embodiment of the invention, method and conditions as described in Example 1 are used, wherein step f) of recovering of the nucleic acids fraction from the supernatant obtained in step e) is performed by ethanol precipitation.

The nucleic acids fraction recovered in step f) comprises mitochondrial ribonucleic acids (RNA). As shown in Example 2 (FIG. 1), the Saccharomyces cerevisiae mitochondrial nucleic acids fraction (i.e. NA fraction; NA-B2 fraction) is RNAse-sensitive. As shown in Example 3 (Table 3), the biological properties of the Saccharomyces cerevisiae mitochondrial nucleic acids fraction (i.e. NA fraction; NA-B2 fraction) are abolished in presence of RNAse.

With this regard, the nucleic acids comprised in the Saccharomyces cerevisiae mitochondrial nucleic acids fraction of the present invention are preferably RNA.

The Saccharomyces cerevisiae mitochondrial nucleic acids fraction of the invention (i.e. NA fraction; NA-B2 fraction) is able to bind to human TLRs. The one skilled in the art is able to determine the ability of a nucleic acid to bind to TLRs by using techniques available in the art such those described in Example 3. In a more preferred embodiment of the invention, the Saccharomyces cerevisiae mitochondrial nucleic acids fraction of the invention is able to bind to human TLR3. TLR4 and TLR7 as described in Example 3.

The Saccharomyces cerevisiae mitochondrial nucleic acids fraction of the invention (i.e. NA fraction; NA-B2 fraction) is intended to orient the immune response toward a Th1 type response directed against an antigen. More particularly, the Saccharomyces cerevisiae mitochondrial nucleic acids fraction of the invention is intended to induce the production of gamma interferon (IFN-γ), interleukin-2 (IL-2) and/or interleukin-12 (IL-12) directed against an antigen. The one skilled in the art is able to determine the ability of a nucleic acid to induce the production of gamma interferon (IFN-γ), interleukin-2 (IL-2) and interleukin-12 (IL-12) by using techniques available in the art such as those described in Example 4 and Example 6. In a more preferred embodiment of the invention, the Saccharomyces cerevisiae mitochondrial nucleic acids fraction of the invention is intended to induce the production of:
gamma interferon (IFN-γ) as described in Example 4 and Example 6, and respectively shown in FIG. 2 and FIG. 4;
interleukin-12 (IL-12) as described in Example 6 and shown in FIG. 5.

As described in Example 6 and shown in FIG. 6, the Saccharomyces cerevisiae mitochondrial nucleic acids fraction of the invention is not capable to induce the production of alpha interferon (IFN-α).

As used throughout the entire application, “antigen” refers to a molecule containing one or more epitopes that will stimulate a host's immune system to make a humoral and/or cellular antigen-specific response. The term is used interchangeably with the term “immunogen”. Antibodies such as anti-idiotype antibodies, or fragments thereof, and synthetic peptide mimotopes, which can mimic an antigen or antigenic determinant, are also captured under the definition of antigen as used herein.

According to the present invention, the antigen is preferably chosen from the group consisting of a tumor associated antigen, an antigen specific to an infectious organism and an antigen specific to an allergen.

According to a first embodiment of the invention, the antigen is tumour associated antigen. As used throughout the entire application, “tumour associated antigen” (TAA) refers to a molecule that is detected at a higher frequency or density in tumor cells than in non-tumor cells of the same tissue type. Examples of TAA includes but are not limited to CEA, MART-1, MAGE-1, MAGE-3, GP-100, MUC-1 (see for instance WO92/07000; EP554344; U.S. Pat. No. 5,861,381; U.S. Pat. No. 6,054,438; WO98/04727; WO98/37095), MUC-2, pointed mutated ras oncogene, normal or point mutated p53, overexpressed p53, CA-125, PSA, C-erb/B2, BRCA I, BRCA II, PSMA, tyrosinase, TRP-1, TRP-2, NY-ESO-1, TAG72, KSA, HER-2/neu, bcr-abl, pax3-fkhr, ews-fli-1, survivin, syncytin (e.g. syncytin-1, see for instance WO99/02696; WO2007/090967; U.S. Pat. No. 6,312,921), mesothelin and LRP. The sequences of these molecules have been described in the prior art. In a preferred embodiment of the invention, the antigen is the TAA MUC-1. Example 5 describes the use of Saccharomyces cerevisiae mitochondrial nucleic acids fraction of the invention (i.e. NA fraction) and MUC-1 antigen for the preparation of a pharmaceutical composition intended for the treatment of cancers.

According to another embodiment of the invention, the antigen is an antigen specific to an infectious organism. As used throughout the entire application, “antigen specific to an infectious organism” refers an antigen specific to a virus, a bacterium, a fungus or a parasite.

As used throughout the entire application, “virus” comprises but is not limited to Retroviridae, Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyviridae (variola viruses, vaccinia viruses, pox viruses); and Iridoviridae (e.g. African swine fever virus). Viral antigens include for example antigens from hepatitis viruses A, B, C, D & E, HIV, herpes viruses, cytomegalovirus, varicella zoster, papilloma viruses, Epstein Barr virus, influenza viruses, para-influenza viruses, adenoviruses, coxsakie viruses, picorna viruses, rotaviruses, respiratory syncytial viruses, pox viruses, rhinoviruses, rubella virus, papovirus, parvovirus, mumps virus, measles virus. Some non-limiting examples of known viral antigens include the following: antigens specific to HIV-1 such as tat, nef, gp120 or gp160, gp40, p24, gag, env, vif, vpr, vpu, rev or part and/or combinations thereof; antigens specific from human herpes viruses such as gH, gL gM gB gC gK gE or gD or part and/or combinations thereof or Immediate Early protein such asICP27, ICP47, ICP4, ICP36 from HSV1 or HSV2; antigens specific from cytomegalovirus, especially human cytomegalovirus such as gB or derivatives thereof; antigens specific to Epstein Barr virus such as gp350 or derivatives thereof; antigens specific to Varicella Zoster Virus such asgpl, 11, 111 and IE63; antigens specific to a hepatitis virus such as hepatitis B, hepatitis C or hepatitis E virus antigen (e.g. env protein E1 or E2, core protein, NS2, NS3, NS4a, NS4b, NS5a, NS5b, p7, or part and/or combinations thereof of HCV); antigens specific to human papilloma viruses (for example HPV6, 11, 16, 18, e.g. L1, L2, E1. E2, E3, E4, E5, E6, E7, or part and/or combinations thereof); antigens specific to other viral pathogens, such as Respiratory Syncytial virus (e.g F and G proteins or derivatives thereof), parainfluenza virus, measles virus, mumps virus, flaviviruses (e.g. Yellow Fever Virus, Dengue Virus, Tick-borne encephalitis virus, Japanese Encephalitis Virus) or Influenza virus cells (e.g. HA, NP, NA, or M proteins, or part and/or combinations thereof). The present invention encompasses notably the use of any HPV E6 polypeptide which binding to p53 is altered or at least significantly reduced and/or the use of any HPV E7 polypeptide which binding to Rb is altered or at least significantly reduced (MUNGER, et al. Complex formation of human papillomavirus E7 proteins with the retinoblastoma tumor suppressor gene product, The EMBO journal. 1989, vol. 8, no. 13, p. 4099-105; CROOK, et al. Degradation of p53 can be targeted by HPV E6 sequences distinct from those required for p53 binding and trans-activation. Cell. 1991, vol. 67, no. 3, p. 547-56.; HECK, et al. Efficiency of binding the retinoblastoma protein correlates with the transforming capacity of the E7 oncoproteins of the human papillomaviruses. Proc. Natl. Acad. Sci. U.S.A. 1992, vol. 89, no. 10, p. 4442-6.; PHELPS, et al. Structure-function analysis of the human papillomavirus type 16 E7 oncoprotein. Journal of Virology. 1992, vol. 66, no. 4, p. 2418-27). A non-oncogenic HPV-16 E6 variant which is suitable for the purpose of the present invention is deleted of one or more amino acid residues located from approximately position 118 to approximately position 122 (+1 representing the first methionine residue of the native HPV-16 E6 polypeptide), with a special preference for the complete deletion of residues 118 to 122 (CPEEK). Anon-oncogenic HPV-16 E7 variant which is suitable for the purpose of the present invention is deleted of one or more amino acid residues located from approximately position 21 to approximately position 26 (+1 representing the first amino acid of the native HPV-16 E7 polypeptide, with a special preference for the complete deletion of residues 21 to 26 (DLYCYE). According to a preferred embodiment, the one or more HPV-16 early polypeptide(s) in use in the invention is/are further modified so as to improve MHC class I and/or MHC class II presentation, and/or to stimulate anti-HPV immunity. HPV E6 and E7 polypeptides are nuclear proteins and it has been previously shown that membrane presentation permits to improve their therapeutic efficacy (see for example WO 99/03885). Thus, it may be advisable to modify at least one of the HPV early polypeptide(s) so as to be anchored to the cell membrane. Membrane anchorage can be easily achieved by incorporating in the HPV early polypeptide a membrane-anchoring sequence and if the native polypeptide lacks it a secretory sequence (i.e. a signal peptide). Membrane-anchoring and secretory sequences are known in the art. Briefly, secretory sequences are present at the N-terminus of the membrane presented or secreted polypeptides and initiate their passage into the endoplasmic reticulum (ER). They usually comprise 15 to 35 essentially hydrophobic amino acids which are then removed by a specific ER-located endopeptidase to give the mature polypeptide. Membrane-anchoring sequences are usually highly hydrophobic in nature and serves to anchor the polypeptides in the cell membrane (see for example BRANDEN, et al. Introduction to protein structure. NY GARLAND, 1991. p. 202-14). The choice of the membrane-anchoring and secretory sequences which can be used in the context of the present invention is vast. They may be obtained from any membrane-anchored and/or secreted polypeptide comprising it (e.g. cellular or viral polypeptides) such as the rabies glycoprotein, of the HIV virus envelope glycoprotein or of the measles virus F protein or may be synthetic. The membrane anchoring and/or secretory sequences inserted in each of the early HPV-16 polypeptides used according to the invention may have a common or different origin. The preferred site of insertion of the secretory sequence is the N-terminus downstream of the codon for initiation of translation and that of the membrane-anchoring sequence is the C-terminus, for example immediately upstream of the stop codon. The HPV E6 polypeptide in use in the present invention is preferably modified by insertion of the secretory and membrane-anchoring signals of the measles F protein. The HPV E7 polypeptide in use in the present invention is preferably modified by insertion of the secretory and membrane-anchoring signals of the rabies glycoprotein. With this regard, in a preferred embodiment of the invention, the antigen is an antigen specific to the Human Papilloma Virus (HPV), preferably an antigen specific to HPV-16 or/and HPV-18, and more preferably an antigen selected from the group consisting of E6 early coding region of HPV-16 or/and HPV-18. E7 early coding region of HPV-16 or/and HPV-18 and part or combination thereof. Example 4 describes the use of Saccharomyces cerevisiae mitochondrial nucleic acids fraction of the invention (i.e. NA fraction; NA-B2 fraction) and HPV16 E7 antigen for the preparation of a pharmaceutical composition intended to orient the immune response towards a Th1 type response against HPV16 E7 antigen.

As used throughout the entire application, “bacterium” comprises gram positive and gram negative bacterium. Gram positive bacterium includes, but is not limited to, Pasteurella species, Staphylococci species, and Streptococcus species. Gram negative bacterium includes, but is not limited to, Escherichia coli, Pseudomonas species, and Salmonella species. Specific examples of infectious bacterium includes but is not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringens, Clostridium tetani, Enterobacter aerogenes, Klebsiella pneumoniae, PastureIla multocida, Bacteroides sp., Fusobacterium nucleatum, Streptobacillus moniliformis, Treponema pallidium, Treponema pertenue, Leptospira, Rickettsia, and Actinomyces israelli.

As used throughout the entire application, “fungus” includes, but is not limited to, Cryptococcus neoformans, Histoplasma capsulatum, Coccidioides immitis, Blastomyces dermatitidis, Chlamydia trachomatis and Candida albicans.

As used throughout the entire application, “parasite” includes, but is not limited to the following genuses: Plasmodium (e.g. Plasmodium falciparum, Plasmodium malariae, Plasmodium spp., Plasmodium ovale or Plasmodium vivax), Babesia (e.g. Babesia microti, Babesia spp. or Babesia divergens), Leishmania (e.g. Leishmania tropica, Leishmania spp., Leishmania braziliensis or Leishmania donovani), Trypanosoma (e.g. Trypanosoma gambiense, Trypanosoma spp., Trypanosoma rhodesiense that causes African sleeping sickness or Trypanosoma cruzi that causes Chagas' disease) and Toxoplasma (e.g. Toxoplasma gondii).

As used throughout the entire application, “allergen” refers to a substance that can induce an allergic or asthmatic response in a susceptible subject. Allergens include, but are not limited to pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (e.g. Felis domesticus); Ambrosia (e.g. Ambrosia artemiisfolia; Lolium (e.g. Lolium perenne or Lolium multiflorum); Cryptomeria (e.g. Cryptomeria japonica); Alternaria (e.g. Alternaria alternata); Alder; Alnus (e.g. Alnus gultinoasa); Betula (e.g. Betula verrucosa); Quercus (e.g. Quercus alba); Olea (e.g. Olea europa); Artemisia (e.g. Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica or Cupressus macrocarpa); Juniperus (e.g. Juniperus sabinoides, Juniperus virginiana, Juniperus communis or Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

According to the invention, the antigen is preferably chosen from the group consisting of a peptide, a nucleic acid (e.g. DNA or RNA, or hybrids thereof), a lipid, a lipopeptide and a saccharide (e.g. oligosaccharide or polysaccharide). The antigen may also be any compound capable of specifically directing the immune response toward a Th1 type response directed against an antigen chosen from the group consisting of a tumor associated antigen, an antigen specific to an infectious organism or an antigen specific to an allergen.

According to a preferred embodiment of the invention, the antigen is comprised in a vector. According to the present invention, the vector is preferably selected from a plasmid or a viral vector.

With regard to a plasmid, it is possible to envisage for instance a plasmid obtained from pBR322 (Gibco BRL), pUC (Gibco BRL), pBluescript (Stratagene), pREP4, pCEP4 (Invitrogene) or p Poly (LATHE, et al. Plasmid and bacteriophage vectors for excision of intact inserts. Gene. 1987, vol. 57, no. 2-3, p. 193-201). In a general manner, plasmids are known to the skilled person and, while a number of them are available commercially (such as for instance the plasmids previously mentioned), it is also possible to modify them or to construct them using the techniques of genetic manipulation. Preferably, a plasmid which is used in the context of the present invention contains an origin of replication which ensures that replication is initiated in a producer cell and/or a host cell (for example, the ColE1 origin will be chosen for a plasmid which is intended to be produced in E. coli and the oriP/EBNA1 system will be chosen if it desired that the plasmid should be self-replicating in a mammalian host cell. LUPTON, et al. Mapping genetic elements of Epstein-Barr virus that facilitate extrachromosomal persistence of Epstein-Barr virus-derived plasmids in human cells. Molecular and cellular biology. 1985, vol. 5, no. 10, p. 2533-42.; YATES, et al. Stable replication of plasmids derived from Epstein-Barr virus in various mammalian cells. Nature. 1985, vol. 313, no. 6005, p. 812-5). The plasmid can additionally comprise a selection gene which enables the transfected cells to be selected or identified (complementation of an auxotrophic mutation, gene encoding resistance to an antibiotic, etc.). Naturally, the plasmid can contain additional elements which improve its maintenance and/or its stability in a given cell (cer sequence, which promotes maintenance of a plasmid in monomeric form (SUMMERS, et al. Multimerization of high copy number plasmids causes instability: ColE1 encodes a determinant essential for plasmid monomerization and stability. Cell. 1984, vol. 36, no. 4, p. 1097-103., sequences for integration into the cell genome).

With regard to a viral vector, it is possible to envisage for instance a viral vector which is obtained from a poxvirus, from an adenovirus, from a retrovirus, from a herpesvirus, from an alphavirus, from a foamy virus or from an adenovirus-associated virus. It is possible to use replication competent or replication deficient viral vectors. A “Replication-competent viral vector” refers to a viral vector capable of replicating in a host cell in the absence of any trans-complementation. A “Replication deficient viral vector” refers to a viral vector that, without some form of trans-complementation, is not capable of replicating in a host cell. Preference will be moreover given to using a vector which does not integrate. In this respect, adenoviral vectors and vectors obtained from poxvirus are very particularly suitable for implementing the present invention.

In a preferred embodiment of the invention, the viral vector is obtained from a poxvirus, preferably from a Vaccinia virus (VV) and more preferably from a modified vaccinia virus Ankara (MVA), or derivatives thereof. “Derivatives” refer to viruses showing essentially the same replication characteristics as the deposited strain but showing differences in one or more parts of its genome.

As used throughout in the entire application, “Vaccinia virus” (VV) includes but is not limited to the VV strains Dairen 1, IHD-J, L-IPV, LC16M8, LC16MO, Lister, LIVP, Tashkent, WR 65-16, Wyeth, Ankara, Copenhagen, Tian Tan, Western Reserve (WR) and derivatives thereof such as for instance VV comprising a defective F2L gene (see WO2009/065547) and VV comprising a defective I4L and/or F4L gene (see WO2009/065546). The VV contains a large duplex DNA genome (187 kilobase pairs) and is a member of the only known family of DNA viruses that replicates in the cytoplasm of infected cells. The VV is fully described in European patent EP83286. The genome of the VV strain Copenhagen has been mapped and sequenced (Goebel et al., 1990, Virol. 179, 247-266 and 517-563; Johnson et al., 1993, Virol. 196, 381-401).

As used throughout in the entire application, “Modified Vaccinia virus Ankara (MVA)” refers to the highly attenuated VV virus generated by 516 serial passages on CEFs of the Ankara strain of VV (CVA) (Mayr, A., et al. Infection 3, 6-14, 1975) and derivatives thereof. The MVA virus was deposited before Collection Nationale de Cultures de Microorganismes (CNCM) under depositary N⁶⁰² I-721. MVA vectors and methods to produce such vectors are fully described in European patents EP 83286 A and EP 206920 A, in the international application WO 07/147,528 as well as in SUTTER, et al. Nonreplicating vaccinia vector efficiently expresses recombinant genes. Proc. Natl. Acad. Sci. U.S.A. 1992, vol. 89, no. 22, p. 10847-51. The genome of the MVA has been mapped and sequenced (Antoine et al., 1998, Virol. 244, 365-396). According to a more preferred embodiment, the antigen may be inserted in deletion I, II, III, IV, V and VI of the MVA vector and even more preferably in deletion III (MEYER, et al. Mapping of deletions in the genome of the highly attenuated vaccinia virus MVA and their influence on virulence. The Journal of general virology. 1991, vol. 72, no. Pt5, p. 1031-8; SUTTER, et al. A recombinant vector derived from the host range-restricted and highly attenuated MVA strain of vaccinia virus stimulates protective immunity in mice to influenza virus. Vaccine. 1994, vol. 12, no. 11, p. 1032-40). Example 5 describes the use of Saccharomyces cerevisiae mitochondrial nucleic acids fraction of the invention (i.e. NA fraction) and MUC-1 antigen for the preparation of a pharmaceutical composition intended for the treatment of cancers, wherein MUC-1 antigen is comprised in a MVA vector.

In another preferred embodiment of the invention, the viral vector is obtained from an adenovirus, an adenovirus-associated virus, a retrovirus, a herpesvirus, an alphavirus or a foamy virus, or a derivative thereof.

Adenoviral vector used according to the present invention is preferably an adenoviral vector which lacks all or part of at least one region which is essential for replication and which is selected from the E1 E2, E4 and L1 L5 regions in order to avoid the vector being propagated within the host organism or the environment. A deletion of the E1 region is preferred. However, it can be combined with (an) other modification(s)-/deletion(s) affecting, in particular, all or part of the E2. E4 and/or L1-L5 regions, to the extent that the defective essential functions are complemented in trans by means of a complementing cell line and/or a helper virus. In this respect, it is possible to use second-generation vectors of the state of the art (see, for example, international applications WO 94/28152 and WO 97/04119). By way of illustration, deletion of the major part of the E1 region and of the E4 transcription unit is very particularly advantageous. For the purpose of increasing the cloning capacities, the adenoviral vector can additionally lack all or part of the non essential E3 region. According to another alternative, it is possible to make use of a minimal adenoviral vector which retains the sequences which are essential for encapsidation, namely the 5′ and 3′ ITRs (Inverted Terminal Repeat), and the encapsidation region. The various adenoviral vectors, and the techniques for preparing them, are known (see, for example. GRAHAM, et al. Methods in molecular biology. Edited by MURREY. The human press inc, 1991. p. 109-128). The origin of the adenoviral vector according to the invention can vary both from the point of view of the species and from the point of view of the serotype. The vector can be obtained from the genome of an adenovirus of human or animal (canine, avian, bovine, murine, ovine, porcine, simian, etc.) origin or from a hybrid which comprises adenoviral genome fragments of at least two different origins. More particular mention may be made of the CAV-I or CAV-2 adenoviruses of canine origin, of the DAV adenovirus of avian origin or of the Bad type 3 adenovirus of bovine origin (ZAKHARCHUK, et al. Physical mapping and homology studies of egg drop syndrome (EDS-76) adenovirus DNA. Archives of virology. 1993, vol. 128, no. 1-2, p. 171-6.; SPIBEY, et al. Molecular cloning and restriction endonuclease mapping of two strains of canine adenovirus type 2. The Journal of general virology, 1989, vol. 70, no. Pt 1, p. 165-72; JOUVENNE, et al. Cloning, physical mapping and cross-hybridization of the canine adenovirus types 1 and 2 genomes. Gene. 1987, vol. 60, no. 1, p. 21-8; MITTAL, et al. Development of a bovine adenovirus type 3-based expression vector. The Journal of general virology. 1995, vol. 76, no. Pt 1, p. 93-102). However, preference will be given to an adenoviral vector of human origin which is preferably obtained from a serotype C adenovirus, in particular a type 2 or 5 serotype C adenovirus. Replication competent adenoviral vectors may also be used according to the present invention. These replication competent adenoviral vectors are well known by the one skilled in the art. Among these, adenoviral vectors deleted in the E1b region coding the 55 kD P53 inhibitor, as in the ONYX-015 virus (BISCHOFF, et al. An adenovirus mutant that replicates selectively in p53-deficient human tumor cells. Science. 1996, vol. 274, no. 5286, p. 373-6; HEISE, et al. An adenovirus E1A mutant that demonstrates potent and selective systemic anti-tumoral efficacy. Nature Medicine. 2000, vol. 6, no. 10, p. 1134-9; WO 94/18992), are particularly preferred. Accordingly, this virus can be used to selectively infect and kill p53-deficient neoplastic cells. A person of ordinary skill in the art can also mutate and disrupt the p53 inhibitor gene in adenovirus 5 or other viruses according to established techniques. Adenoviral vectors deleted in the E1A Rb binding region can also be used in the present invention. For example, Delta24 virus which is a mutant adenovirus carrying a 24 base pair deletion in the E1A region (FUEYO, et al. A mutant oncolytic adenovirus targeting the Rb pathway produces anti-glioma effect in vivo. Oncogene, 2000, vol. 19, no. 1, p. 2-12). Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell. The adenoviral vectors according to the present invention can be generated in vitro in Escherichia coli (E. coli) by ligation or homologous recombination (see, for example, international application WO 96/17070) or else by recombination in a complementing cell line.

Retroviruses have the property of infecting, and in most cases integrating into, dividing cells and in this regard are particularly appropriate for use in relation to cancer. A recombinant retrovirus according to the invention generally contains the LTR sequences, an encapsidation region and the nucleotide sequence according to the invention, which is placed under the control of the retroviral LTR or of an internal promoter such as those described below. The recombinant retrovirus can be obtained from a retrovirus of any origin (murine, primate, feline, human, etc.) and in particular from the MOMuLV (Moloney murine leukemia virus), MVS (Murine sarcoma virus) or Friend murine retrovirus (Fb29). It is propagated in an encapsidation cell line which is able to supply in trans the viral polypeptides gag, pol and/or env which are required for constituting a viral particle. Such cell lines are described in the literature (PA317, Psi CRIP GP+Am-12 etc.). The retroviral vector according to the invention can contain modifications, in particular in the LTRs (replacement of the promoter region with a eukaryotic promoter) or the encapsidation region (replacement with a heterologous encapsidation region, for example the VL3O type) as described in U.S. Pat. No. 5,747,323.

According to the present invention, the vector further comprises the elements necessary for the expression of the antigen when said antigen is a nucleic acid. The elements necessary for the expression may consist of all the elements which enable nucleic acid sequences to be transcribed into RNA and the mRNA to be translated into polypeptide. These elements comprise, in particular, a promoter which may be regulable or constitutive. Naturally, the promoter is suited to the chosen vector and the host cell. Examples which may be mentioned are the eukaryotic promoters of the PGK (phosphoglycerate kinase), MT (metallothionein; MCIVOR. Human purine nucleoside phosphorylase and adenosine deaminase: gene transfer into cultured cells and murine hematopoietic stem cells by using recombinant amphotropic retroviruses. Molecular and cellular biology. 1987, vol. 7, no. 2, p. 838-46), α-1 antitrypsin. CFTR, surfactant, immunoglobulin, actin (TABIN, et al. Adaptation of a retrovirus as a eucaryotic vector transmitting the herpes simplex virus thymidine kinase gene. Molecular and cellular biology. 1982, vol. 2, no. 4, p. 426-36.) and SRα (TAKEBE, et al. SR alpha promoter: an efficient and versatile mammalian cDNA expression system composed of the simian virus 40 early promoter and the R-U5 segment of human T-cell leukemia virus type 1 long terminal repeat. Molecular and cellular biology. 1988, vol. 8, no. 1, p. 466-72.) genes, the early promoter of the SV40 virus (Simian virus), the LTR of RSV (Rous sarcoma virus), the HSV-I TK promoter, the early promoter of the CMV virus (Cytomegalovirus), the p7.5K pH5R, pK1L, p28 and p11 promoters of the vaccinia virus, and the E1A and MLP adenoviral promoters. The promoter can also be a promoter which stimulates expression in a tumor or cancer cell. Particular mention may be made of the promoters of the MUC-I gene, which is overexpressed in breast and prostate cancers (CHEN, et al. Breast cancer selective gene expression and therapy mediated by recombinant adenoviruses containing the DF3/MUC1 promoter. The Journal of clinical investigation. 1995, vol. 96, no. 6, p. 2775-82.), of the CEA (standing for carcinoma embryonic antigen) gene, which is overexpressed in colon cancers (SCHREWE, et al. Cloning of the complete gene for carcinoembryonic antigen: analysis of its promoter indicates a region conveying cell type-specific expression. Molecular and cellular biology. 1990, vol. 10, no. 6, p. 2738-48.) of the tyrosinase gene, which is overexpressed in melanomas (VILE, et al. Use of tissue-specific expression of the herpes simplex virus thymidine kinase gene to inhibit growth of established murine melanomas following direct intratumoral injection of DNA. Cancer res. 1993, vol. 53, no. 17, p. 3860-4.), of the ERBB-2 gene, which is overexpressed in breast and pancreatic cancers (HARRIS, et al. Gene therapy for cancer using tumour-specific prodrug activation. Gene therapy. 1994, vol. 1, no. 3, p. 170-5.) and of the α-fetoprotein gene, which is overexpressed in liver cancers (KANAI, et al. In vivo gene therapy for alpha-fetoprotein-producing hepatocellular carcinoma by adenovirus-mediated transfer of cytosine deaminase gene. Cancer res. 1997, vol. 57, no. 3, p. 461-5). The cytomegalovirus (CMV) early promoter is very particularly preferred. However, when a vector deriving from a Vaccinia virus (as for example an MVA vector) is used, the promoter of the thymidine kinase 7.5K gene is particularly preferred. The necessary elements can furthermore include additional elements which improve the expression of nucleotide sequence according to the invention or its maintenance in the host cell. Intron sequences, secretion signal sequences, nuclear localization sequences, internal sites for the reinitiation of translation of IRES type, transcription termination poly A sequences, tripartite leaders and origins of replication may in particular be mentioned. These elements are known to the skilled person.

The pharmaceutical compositions (and more particularly the adjuvant compositions and the vaccine compositions) according to the invention may further comprise one or more agent which improves the transfectional efficiency and/or the stability of the Saccharomyces cerevisiae mitochondrial nucleic acids fraction and/or the antigen. Said agents are preferably selected from the group consisting of lipid, liposome, submicron oil-in-water emulsion, microparticle, ISCOMs and polymer. The various components of the compositions can be present in a wide range of ratios. For instance, the Saccharomyces cerevisiae mitochondrial nucleic acids fraction of the invention and the agent which improves the transfectional efficiency and/or the stability of the Saccharomyces cerevisiae mitochondrial nucleic acids fraction and/or the antigen can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

As used throughout the entire application, “lipid” comprises neutral, zwitterionic, anionic and/or cationic lipids. Lipids include, but are not limited to phospholipids (e.g. natural or synthetic phosphatidylcholines, phosphatidylethanolamines or phosphatidylserines), glycerides (e.g. diglycerides or triglycerides), cholesterol, ceramides or cerebrosides. Preferred lipids are cationic lipids. Various cationic lipids are known in the art and some are commercially available (e.g. BALASUBRAMANIAM et al. (1996) Gene Ther., 3:163-172; GAO and HUANG (1995) Gene Ther, 2:7110-7122; U.S. Pat. No. 4,897,355; EP 901463 B patent and more preferably pcTG90). In a preferred embodiment of the invention, the lipid is a cationic lipid and more preferably a cationic lipids as described in EP 901463 B patent and even more preferably pcTG90 as described in EP 901463 B patent. The Saccharomyces cerevisiae mitochondrial nucleic acids fraction of the invention and the lipid can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

As used throughout the entire application, “liposome” refers to a vesicle surrounded by a bilayer formed of components usually including lipids optionally in combination with non-lipidic components (such as for instance stearylamine). The liposome forming components used to form the liposomes may include neutral, zwitterionic, anionic and/or cationic lipids. Preferred liposomes are cationic liposomes. Cationic liposomes are widely documented in the literature which is available to the skilled person and some are commercially available (e.g. FELGNER, et al. Cationic liposome mediated transfection. Proceedings of the Western Pharmacology Society. 1989, vol. 32, p. 115-21.; HODGSON, et al. Virosomes: cationic liposomes enhance retroviral transduction. Nature biotechnology. 1996, vol. 14, no. 3, p. 339-42.; REMY, et al. Gene transfer with a series of lipophilic DNA-binding molecules. Bioconjugate chemistry. 1994, vol. 5, no. 6, p. 647-54). Cationic liposomes (as used throughout the entire application) include, but are not limited to dioleoyl phosphatidylethanolamine (DOPE), N-[1-(2,3-dioleyloxy)propyl]-N,N,N-trimethylammonium chloride (DOTMA), 1,2-bis(oleoyloxy)-3-(trimethylammonio)propane (DOTAP), 1,2-bis(hexadecyloxy)-3-trimethylaminopropane (BisHOP), 3[beta][N—(N′N′-dimethylaminoethane)-carbamyl]cholesterol (DC-Chol) or liposomal amphotericin-B (which is commercially available under the trademark Ambisome® from Gilead Sciences). In a preferred embodiment of the invention, the liposome is a cationic liposome, more preferably selected from dioleoyl phosphatidylethanolamine (DOPE), N-[1-(2,3-dioleyloxy)propyl]-N,N,N-trimethylammonium chloride (DOTMA) and liposomal amphotericin-B or combination thereof. The Saccharomyces cerevisiae mitochondrial nucleic acids fraction of the invention and the liposome can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

Liposomal amphotericin-B is commercially available under e.g. the trademark Ambisome® (Gilead Sciences). According to a preferred embodiment of the invention, the Saccharomyces cerevisiae mitochondrial nucleic acids fraction (i.e. NA-B2 fraction) and Ambisome® are preferably used at a ration from about 1:3 to 1:1 (v/v); 1:100 (w/w) as described in Example 2.

A preferred combination of cationic liposomes according to the invention is dioleoyl phosphatidylethanolamine (DOPE) and N-[1-(2,3-dioleyloxy)propyl]-N,N,N-trimethylammonium chloride (DOTMA). Dioleoyl phosphatidylethanolamine (DOPE) and N-[1-(2,3-dioleyloxy)propyl]-N,N,N-trimethylammonium chloride (DOTMA) at a ration of 1:1 (w/w) is commercially available under the trademark Lipofectin® (Invitrogen, Cat. No. 18292-011 or Cat. No. 18292-037). According to a preferred embodiment of the invention, the Saccharomyces cerevisiae mitochondrial nucleic acids fraction (i.e. NA fraction; NA-B2 fraction) and Lipofectin® are preferably at a ration of 1:1 (v/v and/or w/w) as described in Example 1 (NA fraction) and Example 2 (NA-B2 fraction). Another preferred combination according to the invention is dioleoyl phosphatidylethanolamine (DOPE). N-[1-(2,3-dioleyloxy)propyl]-N,N,N-trimethylammonium chloride (DOTMA) and liposomal amphotericin-B. The person skilled in the art is able to determine which ratio between the Saccharomyces cerevisiae mitochondrial nucleic acids fraction of the invention. Lipofectin® and liposomal amphotericin-B are the most appropriate.

As used throughout the entire application, “submicron oil-in-water emulsion” comprises non-toxic, metabolizable oils and commercial emulsifiers. Non-toxic, metabolizable oils include, but are not limited to vegetable oils, fish oils, animal oils or synthetically prepared oils. Commercial emulsifiers include, but are not limited to sorbitan-based non-ionic surfactant (e.g. sorbitan trioleate or polyoxyethylenesorbitan monooleate) or polyoxyethylene fatty acid ethers derived from e.g. lauryl, acetyl, stearyl and oleyl alcohols. Submicron oil-in-water emulsions are widely documented in the literature which is available to the skilled person (e.g. WO 90/14837; TAMILVANAN S., Oil-in-water lipid emulsions: implications for parenteral and ocular delivering systems, Prog Lipid Res. 2004 November; 43(6):489-533). The Saccharomyces cerevisiae mitochondrial nucleic acids fraction of the invention and the submicron oil-in-water emulsion can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

As used throughout the entire application, “microparticle” refers to a particle of about 100 nm to about 150 μm in diameter formed from materials that are sterilizable, non-toxic and biodegradable such as, without limitation, poly(α-hydroxy acid) (e.g. poly(lactide) or poly(D,L-lactide-co-glycolide)), polyhydroxybutyric acid, polycaprolactone, polyorthoester, polyanhydride, polyvinyl alcohol and ethylenevinyl acetate. Microparticles are widely documented in the literature which is available to the skilled person (e.g. RAVI KUMAR M. N. V., Nano and microparticles as controlled grud delivery devices, J. Pharm. Pharmaceut. Sci 3(2):234-258, 2000; WO 07/084,418). The Saccharomyces cerevisiae mitochondrial nucleic acids fraction of the invention and the microparticle can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

As used throughout the entire application, “ISCOMs” refers to immunogenic complexes formed between glycosides such as triterpenoid saponins (particularly Quil A) and antigens which contain a hydrophobic region. ISCOMs are widely documented in the literature which is available to the skilled person (e.g. BARR I. J. and GRAHAM F. M., “ISCOMs (immunostimulating complexes): The first decade”, Immunology and Cell Biology (1996) 74, 8-25; WO 9206710). The Saccharomyces cerevisiae mitochondrial nucleic acids fraction of the invention and the ISCOM can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

As used throughout the entire application, “polymer” includes, but is not limited to, polylysine, polyarginine, polyornithine, spermine and spermidine. The Saccharomyces cerevisiae mitochondrial nucleic acids fraction of the invention and the polymer can be used in a ratio (volume/volume (v/v) and/or weight/weight (w/w)) from about 1:200 to 200:1, preferably 1:100 to 100:1, more preferably from about 1:50 to 50:1, even more preferably from about 1:10 to 10:1, even more preferably from about 1:3 to 3:1, and most preferably of about 1:1.

The applicant has surprisingly found that the Saccharomyces cerevisiae mitochondrial nucleic acids fraction of the invention (i.e. NA fraction; NA-B2 fraction) simultaneously administered with liposomal amphotericin-B (i.e. Ambisome®) statistically significantly increase the Th1 type response (i.e. the production of gamma interferon (IFN-γ), interleukin-2 (IL-2) and/or interleukin-12 (IL-12)) compared with the response resulting from the administration of the Saccharomyces cerevisiae mitochondrial nucleic acids fraction of the invention (i.e. NA fraction; NA-B2 fraction) alone and liposomal amphotericin-B (i.e. Ambisome®) alone, wherein the response resulting from the administration of the Saccharomyces cerevisiae mitochondrial nucleic acids fraction of the invention (i.e. NA fraction; NA-B2 fraction) alone is higher than the response resulting from the administration of liposomal amphotericin-B (i.e. Ambisome®) alone. Such an effect is indifferently called (as used throughout the entire application) ‘synergic effect’ or ‘synergistic effect’. The synergic effect resulting from the simultaneous administration of the NA-B2 fraction and liposomal amphotericin-B (i.e. Ambisome®) is described in Example 6 and shown in FIG. 4 (gamma interferon (IFN-γ)) and FIG. 5 (interleukin-12 (IL-12)).

With this regards, the present invention also relates an adjuvant composition with synergic effect comprises:

(i) a Saccharomyces cerevisiae mitochondrial nucleic acids fraction prepared by a method comprising the following steps:
a) culture of Saccharomyces cerevisiae in a culture medium allowing their growth followed by centrifugation of said culture;
b) grinding of the Saccharomyces cerevisiae pellet obtained in step a);
c) centrifugation of the mixture obtained in step b);
d) ultracentrifugation of the supernatant obtained in step c);
e) extraction of nucleic acids from the pellet obtained in step d);
f) recovering of the nucleic acids fraction from the supernatant obtained in step e); and
   (ii) liposomal amphotericin-B.

With this regards, the present invention also relates a vaccine composition with synergic effect comprises:

(i) a Saccharomyces cerevisiae mitochondrial nucleic acids fraction prepared by a method comprising the following steps:
a) culture of Saccharomyces cerevisiae in a culture medium allowing their growth followed by centrifugation of said culture;
b) grinding of the Saccharomyces cerevisiae pellet obtained in step a);
c) centrifugation of the mixture obtained in step b);
d) ultracentrifugation of the supernatant obtained in step c);
e) extraction of nucleic acids from the pellet obtained in step d);
f) recovering of the nucleic acids fraction from the supernatant obtained in step e);
   (ii) liposomal amphotericin-B; and
   (iii) an antigen.

The applicant has also surprisingly found that the Saccharomyces cerevisiae mitochondrial nucleic acids fraction of the invention (i.e. NA fraction; NA-B2 fraction) simultaneously administered with dioleoyl phosphatidylethanolamine (DOPE) and N-[1-(2,3-dioleyloxy)propyl]-N,N,N-trimethylammonium chloride (DOTMA) Lipofectin®) statistically significantly increase the Th1 type response (i.e. the production of gamma interferon (IFN-γ), interleukin-2 (IL-2) and/or interleukin-12 (IL-12)) compared with the response resulting from the administration of the Saccharomyces cerevisiae mitochondrial nucleic acids fraction of the invention (i.e. NA fraction; NA-B2 fraction) alone and dioleoyl phosphatidylethanolamine (DOPE) and N-[1-(2,3-dioleyloxy)propyl]-N,N,N-trimethylammonium chloride (DOTMA) (i.e. Lipofectin®) alone, wherein the response resulting from the administration of the Saccharomyces cerevisiae mitochondrial nucleic acids fraction of the invention (i.e. NA fraction; NA-B2 fraction) alone is higher than the response resulting from the administration of dioleoyl phosphatidylethanolamine (DOPE) and N-[1-(2,3-dioleyloxy)propyl]-N,N,N-trimethylammonium chloride (DOTMA) (i.e. Lipofectin®) alone. Such an effect is indifferently called (as used throughout the entire application) ‘synergic effect’ or ‘synergistic effect’. The synergic effect resulting from the simultaneous administration of the NA-B2 fraction and dioleoyl phosphatidylethanolamine (DOPE) and N-[1-(2,3-dioleyloxy)propyl]-N,N,N-trimethylammonium chloride (DOTMA) (i.e. Lipofectin®) is described in Example 6 and shown in FIG. 5 (interleukin-12 (IL-12).

With this regards, the present invention also relates an adjuvant composition with synergic effect comprises:

(i) a Saccharomyces cerevisiae mitochondrial nucleic acids fraction prepared by a method comprising the following steps:
a) culture of Saccharomyces cerevisiae in a culture medium allowing their growth followed by centrifugation of said culture;
b) grinding of the Saccharomyces cerevisiae pellet obtained in step a);
c) centrifugation of the mixture obtained in step b);
d) ultracentrifugation of the supernatant obtained in step c);
e) extraction of nucleic acids from the pellet obtained in step d);
f) recovering of the nucleic acids fraction from the supernatant obtained in step e); and
   (ii) dioleoyl phosphatidylethanolamine (DOPE) and N-[1-(2,3-dioleyloxy)propyl]-N,N,N-trimethylammonium chloride (DOTMA).

With this regards, the present invention also relates a vaccine composition with synergic effect comprising:

(i) a Saccharomyces cerevisiae mitochondrial nucleic acids fraction prepared by a method comprising the following steps:
a) culture of Saccharomyces cerevisiae in a culture medium allowing their growth followed by centrifugation of said culture;
b) grinding of the Saccharomyces cerevisiae pellet obtained in step a);
c) centrifugation of the mixture obtained in step b);
d) ultracentrifugation of the supernatant obtained in step c);
e) extraction of nucleic acids from the pellet obtained in step d);
f) recovering of the nucleic acids fraction from the supernatant obtained in step e);
   (ii) dioleoyl phosphatidylethanolamine (DOPE) and N-[1-(2,3-dioleyloxy)propyl]-N,N,N-trimethylammonium chloride (DOTMA); and
   (iii) an antigen.

The present invention also relates to a kit of part. The kit may be a single container housing all the components (i.e. a Saccharomyces cerevisiae mitochondrial nucleic acids fraction of the invention; an antigen; an agent which improves the transfectional efficiency and/or the stability of the Saccharomyces cerevisiae mitochondrial nucleic acids fraction and/or the antigen) together or it may be multiple containers housing individual dosages of the components, such as a blister pack. The kit also has instructions for timing of administration of the different components. The instructions would direct the subject to take the components at the appropriate time. For instance, the appropriate time for delivery of the components may be as the symptoms occur. Alternatively, the appropriate time for administration of the components may be on a routine schedule such as monthly or yearly. The different components may be administered simultaneously or separately as long as they are administered close enough in time to produce a synergistic immune response.

According to a first preferred embodiment, the kit of part comprises a container containing at least one Saccharomyces cerevisiae mitochondrial nucleic acids fraction of the invention and a container containing at least one antigen, and instructions for timing of administration of said components.

According to another preferred embodiment, the kit of part comprises a container containing at least one Saccharomyces cerevisiae mitochondrial nucleic acids fraction of the invention, a container containing at least one antigen and a container containing at least one agent which improves the transfectional efficiency and/or the stability of the Saccharomyces cerevisiae mitochondrial nucleic acids fraction and/or the antigen (said agent being more preferably liposomal amphotericin-B and/or dioleoyl phosphatidylethanolamine (DOPE) and N-[1-(2,3-dioleyloxy)propyl]-N,N,N-trimethylammonium chloride (DOTMA)), and instructions for timing of administration of said components.

The Saccharomyces cerevisiae mitochondrial nucleic acids fraction of the present invention may be used for the preparation of pharmaceutical compositions (and more particularly adjuvant compositions and vaccine compositions) intended for the prevention and/or treatment of mammals against any disease known to those skilled in the art such as, for instance, cancers, infectious diseases, allergies and/or autoimmune disorders.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for prevention or treatment in the present application include precancerous (e.g. benign), malignant, premetastatic, metastatic, and non-metastatic cells. “Cancers” (as used throughout the entire application) include, but are not limited to lung cancer (e.g. small cell lung carcinomas and non-small cell lung), bronchial cancer, oesophageal cancer, pharyngeal cancer, head and neck cancer (e.g. laryngeal cancer, lip cancer, nasal cavity and paranasal sinus cancer and throat cancer), oral cavity cancer (e.g. tongue cancer), gastric cancer (e.g. stomach cancer), intestinal cancer, gastrointestinal cancer, colon cancer, rectal cancer, colorectal cancer, anal cancer, liver cancer, pancreatic cancer, urinary tract cancer, bladder cancer, thyroid cancer, kidney cancer, carcinoma, adenocarcinoma, skin cancer (e.g. melanoma), eye cancer (e.g. retinoblastoma), brain cancer (e.g. glioma, medulloblastoma and cerebral astrocytoma), central nervous system cancer, lymphoma (e.g. cutaneous B-cell lymphoma, Burkitt's lymphoma, Hodgkin's syndrome and non-Hodgkin's lymphoma), bone cancer, leukaemia, breast cancer, genital tract cancer, cervical cancer (e.g. cervical intraepithelial neoplasia), uterine cancer (e.g. endometrial cancer), ovarian cancer, vaginal cancer, vulvar cancer, prostate cancer, testicular cancer. “Cancers” also refer to virus-induced tumors, including, but is not limited to papilloma virus-induced carcinoma, herpes virus-induced tumors, EBV-induced B-cell lymphoma, hepatitis B-induced tumors, HTLV-1-induced lymphoma and HTLV-2-induced lymphoma. In a preferred embodiment of the invention, the Saccharomyces cerevisiae mitochondrial nucleic acids fraction of the present invention may be used for the preparation of pharmaceutical compositions intended for the prevention and/or treatment of mammals against kidney cancer as described in Example 5.

As used throughout the entire application, “infectious diseases” refer to any disease that is caused by an infectious organism. Infectious organisms include, but are not limited to, viruses (e.g. single stranded RNA viruses, single stranded DNA viruses, human immunodeficiency virus (HIV), hepatitis A, B, and C virus, herpes simplex virus (HSV), cytomegalovirus (CMV), respiratory syncytial virus (RSV), Epstein-Barr virus (EBV) or human papilloma virus (HPV)), parasites (e.g. protozoan and metazoan pathogens such as Plasmodia species, Leishmania species, Schistosoma species or Trypanosoma species), bacteria (e.g. Mycobacteria in particular, M. tuberculosis, Salmonella, Streptococci, E. coli or Staphylococci), fungi (e.g. Candida species or Aspergillus species), Pneumocystis carinii, and prions. In a preferred embodiment of the invention, the Saccharomyces cerevisiae mitochondrial nucleic acids fraction of the present invention may be used for the preparation of pharmaceutical compositions intended for the prevention and/or treatment of mammals against human papilloma viruses (HPV) as described in Example 4.

As used throughout the entire application, “allergies” refer to any allergy that is caused by an allergen such as for instance allergens previously mentioned according to the present invention.

As used throughout the entire application, “autoimmune disorders” may be categorized into two general types: ‘Systemic autoimmune diseases’ (i.e., disorders that damage many organs or tissues), and ‘localized autoimmune diseases’ (i.e., disorders that damage only a single organ or tissue). However, the effect of ‘localized autoimmune diseases’, can be systemic by indirectly affecting other body organs and systems. ‘Systemic autoimmune diseases’ include but are not limited to rheumatoid arthritis which can affect joints, and possibly lung and skin; lupus, including systemic lupus erythematosus (SLE), which can affect skin, joints, kidneys, heart, brain, red blood cells, as well as other tissues and organs; scleroderma, which can affect skin, intestine, and lungs; Sjogren's syndrome, which can affect salivary glands, tear glands, and joints; Goodpasture's syndrome, which can affect lungs and kidneys; Wegener's granulomatosis, which can affect sinuses, lungs, and kidneys; polymyalgia rheumatica, which can affect large muscle groups, and temporal arteritis/giant cell arteritis, which can affect arteries of the head and neck. ‘Localized autoimmune diseases’ include but are not limited to Type 1 Diabetes Mellitus, which affects pancreas islets; Hashimoto's thyroiditis and Graves' disease, which affect the thyroid; celiac disease. Crohn's diseases, and ulcerative colitis, which affect the gastrointestinal tract; multiple sclerosis (MS) and Guillain-Barre syndrome, which affect the central nervous system; Addison's disease, which affects the adrenal glands; primary biliary sclerosis, sclerosing cholangitis, and autoimmune hepatitis, which affect the liver; and Raynaud's phenomenon, which can affect the fingers, toes, nose, ears.

The pharmaceutical compositions (and more particularly adjuvant compositions and vaccine compositions) comprising the Saccharomyces cerevisiae mitochondrial nucleic acids fraction of the present invention may further comprise a pharmaceutically acceptable carrier. The pharmaceutically acceptable carrier is preferably isotonic, hypotonic or weakly hypertonic and has a relatively low ionic strength, such as for example a sucrose solution. Moreover, such a carrier may contain any solvent, or aqueous or partially aqueous liquid such as nonpyrogenic sterile water. The pH of the pharmaceutical composition is, in addition, adjusted and buffered so as to meet the requirements of use in vivo. The pharmaceutical compositions (and more particularly adjuvant compositions and vaccine compositions) may also include a pharmaceutically acceptable diluent, adjuvant or excipient, as well as solubilizing, stabilizing and preserving agents. For injectable administration, a formulation in aqueous, nonaqueous or isotonic solution is preferred. It may be provided in a single dose or in a multidose in liquid or dry (powder, lyophilisate and the like) form which can be reconstituted at the time of use with an appropriate diluent.

The present invention also relates to a method of orienting in a mammal the immune response toward a Th1 type response directed against an antigen, comprising administering to the mammal an antigen and a Saccharomyces cerevisiae mitochondrial nucleic acids fraction prepared by the method according to the invention. In one embodiment, the method comprises simultaneous administration of the antigen and the Saccharomyces cerevisiae mitochondrial nucleic acids fraction of the invention. Alternatively, the method comprises sequential administration of the antigen and the Saccharomyces cerevisiae mitochondrial nucleic acids fraction of the invention. As used herein, the term “sequential” means that the components are administered to the subject one after another within a timeframe. Thus, sequential administration may permit one component to be administered within some minutes or a matter of hours after the other. For instance, Example 5 describes the use of Saccharomyces cerevisiae mitochondrial nucleic acids fraction of the invention (i.e. NA fraction) and MUC-1 antigen for the preparation of a pharmaceutical composition intended for the treatment of cancers, wherein the Saccharomyces cerevisiae mitochondrial nucleic acids fraction (i.e. NA fraction) is injected one hour later after the MUC-1 antigen.

Administering the pharmaceutical compositions (and more particularly adjuvant compositions and vaccine compositions) of the present invention, and more particularly administering the different components of said compositions (i.e. a Saccharomyces cerevisiae mitochondrial nucleic acids fraction of the invention; an antigen; an agent which improves the transfectional efficiency and/or the stability of the Saccharomyces cerevisiae mitochondrial nucleic acids fraction and/or the antigen) may be accomplished by any means known to the skilled artisan. Preferred routes of administration include but are not limited to intradermal, subcutaneous, oral, parenteral, intramuscular, intranasal, intratumoral, sublingual, intratracheal, inhalation, ocular, vaginal, and rectal. According to a preferred embodiment, the pharmaceutical compositions (and more particularly adjuvant compositions and vaccine compositions) of the invention and more particularly the components of said compositions are delivered subcutaneously or intradermally. According to an even more preferred embodiment of the invention, the antigen and the Saccharomyces cerevisiae mitochondrial nucleic acids fraction of the invention are administered at the same site. For instance, Example 5 describes the use of Saccharomyces cerevisiae mitochondrial nucleic acids fraction of the invention (i.e. NA fraction) and MUC-1 antigen for the preparation of a pharmaceutical composition intended for the treatment of cancers, wherein the Saccharomyces cerevisiae mitochondria: nucleic acids fraction (i.e. NA fraction) and the MUC-1 antigen are administered subcutaneously at the same site.

The administration may take place in a single dose or a dose repeated one or several times after a certain time interval. Desirably, the pharmaceutical compositions and more particularly the components of said pharmaceutical compositions are administered 1 to 10 times at weekly intervals. For instance, Example 5 describes the use of Saccharomyces cerevisiae mitochondrial nucleic acids fraction of the invention (i.e. NA fraction) and MUC-1 antigen for the preparation of a pharmaceutical composition intended for the treatment of cancers, wherein the Saccharomyces cerevisiae mitochondrial nucleic acids fraction (i.e. NA fraction) and the MUC-1 antigen are administered 3 times at weekly intervals.

The dose of administration of the antigen will also vary, and can be adapted as a function of various parameters, in particular the mode of administration; the pharmaceutical composition employed; the age, health, and weight of the host organism; the nature and extent of symptoms; kind of concurrent treatment; the frequency of treatment; and/or the need for prevention or therapy. Further refinement of the calculations necessary to determine the appropriate dosage for treatment is routinely made by a practitioner, in the light of the relevant circumstances.

For general guidance, suitable dosage for a MVA-comprising composition varies from about 10⁴ to 10¹⁰ pfu (plaque forming units), desirably from about 10⁵ and 10⁸ pfu whereas adenovirus-comprising composition varies from about 10⁵ to 10¹³ iu (infectious units), desirably from about 10⁷ and 10¹² iu. A composition based on vector plasmids may be administered in doses of between 10 μg and 20 mg, advantageously between 100 μg and 2 mg. In a preferred embodiment of the invention, the pharmaceutical composition is administered at dose(s) comprising from 5 10⁵ pfu to 5 10⁷ pfu of MVA vector. For instance, Example 5 describes the use of Saccharomyces cerevisiae mitochondrial nucleic acids fraction of the invention (i.e. NA fraction) and MUC-1 antigen for the preparation of a pharmaceutical composition intended for the treatment of cancers, wherein the MUC-1 antigen which is comprised in an MVA vector is administered at 5 10⁷ pfu.

When the use, the method, the adjuvant composition, the vaccine composition or the kit of part according to the invention is for the treatment of cancer, the use, the method, the adjuvant composition, the vaccine composition or the kit of part of the invention CaO be carried out in conjunction with one or more conventional therapeutic modalities (e.g. radiation, chemotherapy and/or surgery). The use of multiple therapeutic approaches provides the patient with a broader based intervention. In one embodiment, the method of the invention can be preceded or followed by a surgical intervention. In another embodiment, it can be preceded or followed by radiotherapy (e.g. gamma radiation). Those skilled in the art can readily formulate appropriate radiation therapy protocols and parameters which can be used (see for example PEREZ. Principles and practice of radiation oncology. 2nd edition. LIPPINCOTT, 1992.; using appropriate adaptations and modifications as will be readily apparent to those skilled in the field).

The present invention further concerns a method for improving the treatment of a cancer patient which is undergoing chemotherapeutic treatment with a chemotherapeutic agent, which comprises co-treatment of said patient along with a method as above disclosed.

The present Invention further concerns a method of improving cytotoxic effectiveness of cytotoxic drugs or radiotherapy which comprises co-treating a patient in need of such treatment along with a method as above disclosed.

When the use, the method, the adjuvant composition, the vaccine composition or the kit of part according to the invention is for the treatment of an infectious disease, the use, the method, the adjuvant composition, the vaccine composition or the kit of part of the invention can be carried out with the use or another therapeutic compounds such as antibiotics, antifungal compounds, antiparasitic compounds and/or antiviral compounds.

The present invention further concerns a method of improving the therapeutic efficacy of an antibiotic, an antifungal, an antiparasitic and/or an antiviral drug which comprises co-treating a patient in need of such treatment along with a method as above disclosed.

In another embodiment, the use, the method, the adjuvant composition, the vaccine composition or the kit of part of the invention is carried out according to a prime boost therapeutic modality which comprises sequential administration of one or more primer composition(s) and one or more booster composition(s). Typically, the priming and the boosting compositions use different vehicles which comprise or encode at least an antigenic domain in common. The priming composition is initially administered to the host organism and the boosting composition is subsequently administered to the same host organism after a period varying from one day to twelve months. The method of the invention may comprise one to ten sequential administrations of the priming composition followed by one to ten sequential administrations of the boosting composition. Desirably, injection intervals are a matter of one week to six months. Moreover, the priming and boosting compositions can be administered at the same site or at alternative sites by the same route or by different routes of administration.

### BRIEF DESCRIPTION OF THE DRAWINGS

FIG. 1: NA fraction, NA-B1 fraction and NA-B2 fraction in agarose gel (1%) in 1×TAE (Tris-Acetate-EDTA) buffer, with or without RNAseA treatment.

FIG. 2: In vivo ELISpot gamma interferon (IFN-γ) resulting from subcutaneous injection (day 0; day 7 and day 14) of HPV16E7 antigen (10 μg) with NA fraction (25 μg) or NA-B2 fraction (0.4 μg).

FIG. 3: Effect of the subcutaneous administration (at day 4, day 11 and day 18) of 5·10⁷ pfu of MVA strain expressing MUC1 antigen and hIL-2 (MVA9931) and (1 h later) NA fraction (50 μg) on the tumor volume of B6D2 mice injected subcutaneously with 3·10⁵ RenCa-MUC-1 cells (at day 1). Effect of the intratumoral (I.T.) administration (at day 4, day 11 and day 18) of NA+Lipofectin® (50 μg+50 μg). Tumor volume was measured twice a week.

FIG. 4: Induction of gamma interferon (IFN-γ) in human immature monocyte-derived dendritic cells (moDCs) treated with NA-B2 fraction (0.4 μg or 1.2 μg), Ambisome® (120 μg) or NA-B2+Ambisome® (0.4 μg+120 μg or 1.2 μg+120 μg).

FIG. 5: Induction of interleukin-12 (IL-12) in human immature moDCs treated with NA-B2 fraction (0.2 μg), Lipofectin® (10 μg), Ambisome® (80 μg, 120 μg or 160 μg), NA-B2+Lipofectin® (0.2 μg+10 μg) and NA-B2+Ambisome® (0.2 μg+120 μg).

FIG. 6: Induction of alpha interferon (IFN-α) in human immature moDCs treated with NA-B2 fraction (0.4 μg or 1.2 μg), Ambisome® (120 μg or 240 μg) and NA-B2+Ambisome® (0.4 μg+120 μg, 0.4 μg+240 μg, 1.2 μg+120 μg or 1.2 μg+240 μg).

### EXAMPLES

To illustrate the invention, the following examples are provided. The examples are not intended to limit the scope of the invention in any way.

### Example 1

### Preparation of the Saccharomyces cerevisiae Mitochondrial Nucleic Acids Fraction

### NA Fraction

An aliquot of frozen Saccharomyces cerevisiae (S.c.) AH109 (Clonetech) was spread on YPG plates composed of 1% yeast extract, 1% Bacto-peptone 2% glucose, 2% agar (BD Sciences) and 100 μg/ml adenine (Fluke 01830-5G). Grown at 28° to 30° C. for two days, an aliquot of S.c. AH109 was taken with a spatula to inoculate 100 ml of liquid YPG/adenine medium poured in a 500 ml vial. After overnight incubation at 28° C. under agitation (200 rpm), 15 ml of this pre-culture were transferred in six 2000 ml vials containing 500 ml YPG/adenine medium, respectively. These cultures (3 litres in total) were incubated overnight at 28° C. under agitation (200 rpm). At an optical density measured at 600 nm (OD₆₀₀) of 2+/−0.5, the culture was centrifuged at 3500 rpm (Sorvall centrifuge, 500 ml tubes) during 15 min at 4° C.

The cell pellets were washed once with distilled water e.g. 1 litre of distilled water per pellet derived from 3-litre culture. After centrifugation (Sorvall, 3500 rpm during 15 min at 4° C.) cell pellets were dissolved in PBS such that the OD₆₀₀ of the resulting suspension was around 100 (e.g. cell pellets derived from 3-litre culture were dissolved in 40 ml PBS). From this step samples were always kept in the cold (4° C.): 30 ml of said cell suspension were transferred in a 125 ml Polyethylene Terephthalate Glycol (PETG) flask and mixed with 30 ml of sterile glass beads (diameter 0.7 mm). The mixture was vortexed (desktop vortex TOP MIX 94323 BIOBLOCK Scientifique) five times at maximum speed for 1 minute alternating with 1 minute incubation on ice. The cell lysate was recovered using a 5 ml glass pipette extended with a blue 1000 μl blue tip to avoid aspiration of glass beads, and was transferred in 50 ml centrifugation tube (Corning) together with 10 ml of PBS used to rinse the glass beads.

Cell lysate was centrifuged at 4000 rpm for 10 min at 4° C. (Sorvall) to pellet the membrane debris as well as the nuclei.

Supernatant obtained was ultra-centrifuged in 12 ml tubes for 90 min at 39000 rpm at 4° C. in a SW40 rotor (105000 g) to pellet the mitochondria. Pellets were dissolved in cold PBS (e.g. pellet obtained from initially 9 litres of S.c. culture was taken up in 100 ml PBS). The resulting mitochondrial fraction was named SN.

The SN fraction obtained was treated with phenol to extract nucleic acids from proteins and lipids. To that, an equal volume of Tris-buffered phenol (Amresco) was added to the suspension, vortexed at max speed for 1 min at room temperature (RT) and centrifuged (e.g. 50 ml Falcon tube centrifuged at 5000 rpm for 10 min at RT in Hareus centrifuge). The aqueous upper phase was isolated and transferred in a new tube. Phenol extraction was repeated three times. Aqueous upper phase recovered after three phenol extractions was then extracted twice with dichloromethane (p.A.; Merck): equal volume of dichloromethane was added and the mixture was vortexed 30 sec at RT and centrifuged (e.g. 50 ml Falcon tube centrifuged at 5000 rpm for 10 min at RT in Hereaus centrifuge). The aqueous phase was recovered and the dichloromethane-treatment was repeated.

Nucleic acids were recovered from the isolated supernatant by ethanol precipitation: 3M sodium acetate pH 5 was added at 1/10 of the supernatant volume as well as 2 volumes of ethanol (abs). After overnight incubation at 4° C. the solution was centrifuged (e.g. 50 ml Falcon tubes in Hareaus centrifuge for 20 min at 4° C.). The pellets were washed with cold 70% ethanol. Before completely dried, pellets were taken up in TE pH 7.5 (e.g. pellets derived from 100 ml suspension obtained in step d) were taken up in 20-25 ml of TE pH 7.5, resulting in nucleic acid concentrations as measured by optical density at 260 nm of around 1 μg/μl). The resulting mitochondrial nucleic acid fraction was named NA fraction.

Three independent large scale preparations of the Saccharomyces cerevisiae nucleic acids fraction (i.e. NA fraction) starting from 9 litres S.c. cultures have been performed according to the described method. The three preparations led to comparable characteristics. The endotoxin levels measured by LAL assay in all of the three preparations were low and comparable (between 0.5 and 0.7 EU/ml).

Preparations of the Saccharomyces cerevisiae nucleic acids fraction (i.e. NA fraction) starting from S.c. W303 (Biochem) have also been performed.

To generate NA fraction-Lipofectin® (that will be tested in the following Examples), the NA fraction (1 μg/μl) was mixed with Lipofectin® (1 μg/μl; Invitrogen, Cat. No. 18292-011 or Cat. No. 18292-037) at a ratio of 1:1 (v:v and w:w).

### Example 2

### Isolation of the Mitochondrial RNA from the NA Fraction

### NA-B2 Fraction

NA fraction prepared according to the method described in Example 1 was run on 1% agarose gel in 1×TAE (Tris-Acetate-EDTA) buffer.

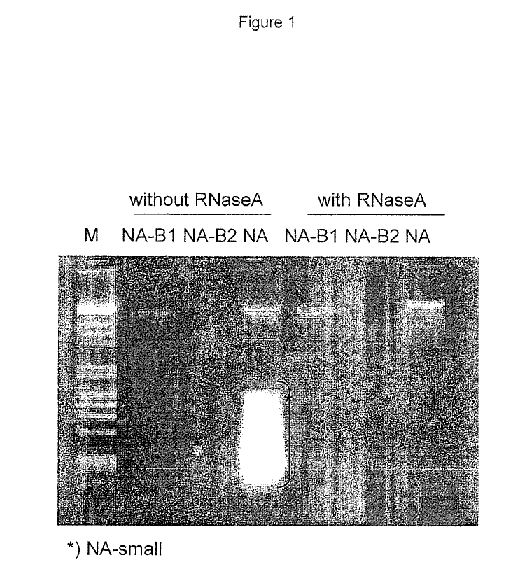

Results as depicted in FIG. 1 show that compared to DNA marker Lambda-HindIII/PhiX174-HaeIII (called M in FIG. 1), three groups of nucleic acids could clearly be distinguished:
(1) a distinct band migrating around 20 Kbp, called NA-B1 fraction;
(2) a distinct band migrating around 4 kbp, called NA-B2 fraction; and
(3) a smear of molecules migrating between 1000 and ˜100 bp, called NA-small fraction.

Purification of NA-B1 fraction, NA-B2 fraction and NA-small fraction was then realized by cutting out the respective bands or groups of bands from agarose gel using mild UV and a scalpel. Excised agarose cubes were transferred in “double-tube constructs” (=a 0.5 ml tube with hole at the bottom applied with a hot needle and with cotton plugged in serving a filter was inserted in 2 ml tube with lid being cut off), frozen at less than −60° C., centrifuged at RT for 15 min in bench-top centrifuge at 5000 rpm (until material is completely thawed) followed by 2 min centrifugation at 14000 rpm. The solution recovered in the lower tube was transferred in new tube. Nucleic acids were precipitated using sodium-acetate and ethanol as described in Example 1. Pellets were taken up in TE pH 7.5. Typically, starting from 3 mg of NA fraction run on agarose gel, ˜8 μg of NA-B2 fraction were recovered, typically dissolved in TE pH 7.5 to a concentration of 20 ng/μl.

NA fraction, NA-B1 fraction and NA-B2 fraction were then run on 1% agarose gel in 1×TAE (Tris-Acetate-EDTA) buffer, with or without RNAseA treatment (100 mg/ml; Qiagen).

Results as depicted in FIG. 1 show that:
(1) NA-B1 fraction turned out to be HaeIII-sensitive and RNAseA-insensitive, demonstrating NA-B1 fraction to be DNA;
(2) NA-B2 fraction and NA-small fraction were HaeIII-insensitive and RNAseA-sensitive, demonstrating these molecules to be RNA.

Same results have been obtained with fractions obtained from S.c. AH109 (Clonetech) and fractions obtained from S.c. W303 (Biochem).

To generate NA-B2 fraction-Lipofectin® (that will be tested in the following Examples), the NA-B2 fraction (20 ng/μl) was mixed with Lipofectin® (1 μg/μl; Invitrogen, Cat. No. 18292-011 or Cat. No. 18292-037) at a ratio of 1:1 (v:v).

To generate NA-B2 fraction-Ambisome® (that will be tested in the following Examples), the NA-B2 fraction (20 ng/μl) was mixed with Ambisome® (4 μg/μl; Gilead Sciences) at a ratio of 1:1 or 1:3 (v:v).

### Example 3

### Ability of NA Fraction and NA-B2 Fraction to Stimulate Human Toll-Like Receptors (TLRs)

Cells: Human embryonic kidney cells 293 (HEK) were stably transfected with plasmids allowing for the constitutive expression of one or two Toll like Receptors of human origin (hTLR). The resulting cell lines 293/hTLR2-CD14, 293/hTLR3, 293/hTLR4-MD2-CD14, 293/hTLR5, 293/hTLR2/6, 293/hTLR7, 293/hTLR8 and 293/hTLR9 were purchased from InvivoGen (San Diego, Calif., USA). All cell lines were cultivated in the presence of Blasticidin S (10 μg/ml, InvivoGen) in Dulbecco's minimal Eagle's medium (DMEM) supplemented with 10% fetal calf serum, 40 μg/ml Gentamycin, 2 mM Glutamine, 1 mM sodium pyruvate (Sigma) and 1× Non Essential Amino acids (NEAA, Gibco). In the case of 293/hTLR2-CD14 and 293/hTLR4-MD2-CD14 Hygromycin B was added to a concentration of 100 μg/ml. All eight cell lines were stably transfected with the NF-kB-inducible reporter plasmid pNiFty (InvivoGen). pNiFty encodes the firefly luciferase gene under control of an engineered ELAM1 promoter which combines five NF-kB sites and the proximal ELAM promoter. Stable transfectants were selected in the presence of 100 μg/ml Zeocin (InvivoGen). The emerging clones “hTLRx-luc” were characterized with respect to EC₅₀ and fold-induction to the respective control TLR ligands. Clones with lowest EC₅₀ and high fold inductions and good/acceptable growth behaviour were chosen. The retained clones and their characteristics are listed in Table 1.

TABLE 1

| | Characteristics | | | | |
|---|---|---|---|---|---|
| | | Fold | Assay conditions | | |
| HeK - 293 | Ligand EC50 | induction | Ligand concentration | Cells/96 | Stability |
| cell line | (InvivoGen) | max/min | 1.8x and >10x EC50 | well | Stability |
| hTLR 2-luc | FSL-1 2.9_{nM} | 157 | 5.2 nM/50 nM (17x) | 1.00E+04 | <p19 |
| hTLR 3-luc | Poly(I:C) 1.8_{ng/ml} | 62 | 5.8 ng/ml/25 ng/ml | 9 × 10+03 | >p19 |
| | | | (14x) | | |
| hTLR 4-luc | LPS 0.9_{ng/ml} | 17 | 1.8 ng/ml/20 ng/ml | 2.5 × 10+03 | <p19 |
| | | | (22x) | | |
| hTLR 5-luc | Flagellin 66.2_{ng/ml} | 392 | 119 ng/ml/1 μg/ml | 1.5 × 10+04 | >p19 |
| | | | (15x) | | |
| hTLR 2/6- | FSL-1 0.64_{nM} | 140 | 1.15 nM/20 nM (31x) | 7.5 × 10+04 | >p19 |
| luc | | | | | |
| hTLR 7-luc | R-848 2.9_{×10-7 M} | 126 | 5 × 10−7 M/5 × 10−6 M | 4 × 10+04 | <p19 |
| | | | (17x) | | |
| hTLR 8-luc | R-848 3_{×10-5 M} | 261 | 5 × 10−5 M/5 × 10−4 M | 6 × 10+04 | >p19 |
| | | | (16x) | | |
| hTLR 9-luc | ODN 2006 0.68_{μM} | 11 | 1.2 μM/10 μM (16x) | 5 × 10+04 | p4 |
| 293-luc-2-8 | Poly(I:C)/LyoVec | 20 | 1 ng/ml/10 ng/ml | 1.00E+04 | |
| | [08 ng/ml] | | | | |

Control cell lines 293-luc-2-8 (293-luc): HEK-293 cells were stably transfected with the NF-kB-inducible reporter plasmid pNiFty2. Stable transfectants were selected in the presence of 100 μg/ml Zeocin (InvivoGen). The positive clone 293-luc-2 was subcloned, clone 293-luc-2-8 was retained. This control cell line was generated to control for TLR-independent stimulation of the NF-kB pathway.

RT-PCR experiments have shown (data no shown) that all cell lines are positive for rig-I (retinoic acid inducible gene 1) and mda-5 (melanoma differentiation antigen 5) messages, being members of the RLH family (KR08001 p75, Renée Brandely, October 2008). In addition it was shown (data no shown) that all cell lines could be stimulated by formulated poly(I:C) “polyICLyoVec” (InvivoGen), being described as MDA-5 ligand by the supplier. This result suggests the functionality of MDA-5 in all cell lines (TLR and control cell line).

In vitro TLR tests—method: Cells diluted in DMEM supplemented with 2% fetal calf serum, 40 μg/ml Gentamycin, 2 mM Glutamine, 1 mM sodium pyruvate (Sigma) and 1×MEM non essential amino acids (NEAA, Gibco) were seeded in 96 well plates. The next day. NA fraction (stock: 1 mg/ml) either alone or in combination with Lipofectin® (as described in Example 1) was added at a concentration of 16 μg/ml and 3-fold serial dilutions thereof. As positive controls, the cell lines were stimulated with a defined amount of their respective reference ligands. The day after stimulation (18-20 hours) later, cells were lysed in 100 μl buffer containing 125 mM Tris pH 7.8, 10 mM EDTA, 5 mM DTT and 5% Triton X-100. Firefly luciferase activity in 10 μl lysate was quantified by integrate measurement of flash luminescence over 1 sec (LB96 P Microlumat, Berthold) after addition of 50 μl luciferase revelation buffer (1× luciferase revelation buffer: 20 mM Tris pH 7.8, 1.07 mM MgCl₂, 2.7 mM MgSO₄, 0.1 mM EDTA, 33.3 mM DTT, 470 μM luciferine 530 μM ATP and 270 μM CoEnzyme A). The resulting relative light units (RLU) were expressed as percentage of induction compared to the control ligand and analyzed with the Graph Pad Prism 4 software using an equation for sigmoid dose response (determination of EC₅₀).

In vitro TLR tests N^{o} 1: Two independent batches of NA fraction were tested (Lot 1: 0.6 EU/ml; Lot 2: 0.77 EU/ml) either alone or in combination with Lipofectin® on TLR cell lines and control cell lines according to the method previously described. The maximal activation expressed in percentage of what was observed with the respective control ligand (see Table 1) is indicated in Table 2.

TABLE 2

| | TLR2 | TLR3 | TLR4 | TLR5 | TLR2/6 | TLR7 | TLR8 | TLR9 | 293-luc |
|---|---|---|---|---|---|---|---|---|---|
| | % act max | % act max | % act max | % act max | % act max | % act max | % act max | % act max | % act max |
| NA (lot1) | 0 | 7 | 14 | 0 | 0 | 8 | 0 | 0 | 0.2 |
| NA (lot2) | 0 | 2 | 5 | 0 | 0 | 7 | 0 | 0 | 1.5 |
| NA(1) + Lipofectin | 24 | 44 | 41 | 13 | 70 | 23 | 0 | 58 | 87 |
| NA(2) + Lipofectin | 35 | 61 | 80 | 17 | 77 | 36 | 0.5 | 67 | 95 |
| Lipofectin | 0 | 1 | 2 | 0 | 0 | 0 | 0 | 0 | not done |
| Herring sperm DNA + Lipofectin | 0 | 0 | 1 | 1 | 0 | 0 | 0 | 3 | not done |

Results depicted in Table 2 show that:
(1) Stimulation is observed with NA fraction in hTLR 3, 4 and 7 (lot 1 as well as lot 2);
(2) Stimulation observed with NA alone is strongly increased when NA was mixed with Lipofectin® (lot 1 as well as lot 2);
(3) Lipofectin® alone or Lipofectin® mixed with herring sperm DNA (1 μg/ml; Sigma) at a ratio of 1:1 (w:w), did not stimulate any of the cell lines.

In vitro TLR tests N^{o} 2: NA-B1 fraction, NA-B2 fraction (1.3 EU/ml) and NA fraction (0.7 EU/ml), treated with RNAseA (100 mg/ml; Qiagen) before adding Lipofectin® were tested on TLR cell lines and control cell lines according to the method previously described. Results are depicted in Table 3.

TABLE 3

| | 293-luc | TLR3 | TLR7 | TLR9 |
|---|---|---|---|---|
| | Maximal | Maximal | Maximal | Maximal |
| | Activation | Activation | Activation | Activation |
| | (%) | (%) | (%) | (%) |
| NA-B1 | 2 | 0 | 0 | 0 |
| NA-B2 | 12 | 54 | 34 | 5 |
| NA | 0 | 24 | 32 | 3 |
| NA-B1 + RNaseA | 0 | 0 | 0 | 0 |
| NA-B2 + RNaseA | 0 | 0 | 1 | 3 |
| NA + RNaseA | 0 | 0 | 1 | 0 |
| NA-B1 + Lipofectin ® | 0 | 0 | 0 | 2 |
| NA-B2 + Lipofectin ® | 89 | 27 | 29 | 54 |
| NA + Lipofectin ® | 132 | 49 | 66 | 36 |
| NA-B1 + RNaseA + | 0 | 0 | 1 | 5 |
| Lipofectin ® | | | | |
| NA-B2 + RNaseA + | 0 | 0 | 1 | 3 |
| Lipofectin ® | | | | |
| NA + RNaseA + | 0 | 0 | 1 | 5 |
| Lipofectin ® | | | | |
| Lipofectin ® | 1 | 0 | 0 | 4 |

Results depicted in Table 3 show that:
(1) NA fraction and NA-B2 fraction as well, without and more so with Lipofectin®, stimulate the tested cell lines including 293-luc;
(2) Both stimulation by NA fraction and NA-Lipofectin® were abolished after pre-treatment of NA fraction with RNaseA. This demonstrates that the active molecule is RNA.
(3) Both stimulation by NA-B2 fraction and NA-B2-Lipofectin® were abolished after pre-treatment of NA-B2 fraction with RNaseA. This demonstrates that the active molecule is RNA.
(4) NA-B1 fraction, with or without Lipofectin®, do not stimulate the TLR cell lines;
(5) Lipofectin® alone has no effect.

Results in terms of HEK-293-TLR cell line stimulation obtained with NA-B2 from AH119 (Clonetech) are comparable to results obtained with NA-B2 from S.c. strain W303 (Biochem).

### Example 4

### Use of NA Fraction or NA-B2 Fraction, and HPV16 E7 Antigen for the Preparation of a Pharmaceutical Composition Intended to Orient the Immune Response Towards a Th1 Type Response Against HPV16 E7 Antigen

Animals model: SPF healthy female C57BL/6 mice were obtained from Charles River (Les Oncins, France). The animals were 6-weeks-old upon arrival. At the beginning of experimentation, they were 7-week-old. The animals were housed in a single, exclusive room, air-conditioned to provide a minimum of 11 air changes per hour. The temperature and relative humidity ranges were within 20° C. and 24° C. and 40 to 70% respectively. Lighting was controlled automatically to give a cycle of 12 hours of light and 12 hours of darkness. Specific pathogen free status was checked by regular control of sentinel animals. Throughout the study the animals had access ad libitum to sterilized diet type RM1 (Dietex France, Saint Gratien). Sterile water was provided ad libitum via bottles.

In vivo ELISpot Gamma Interferon (IFN-γ):

IFN-γ ELIspot assay is a functional test to determine the ability of in vivo primed T cells to secrete IFN-γ upon re-stimulation in vitro with a specific peptide.

Animals were injected 3 times at a one week interval (day 0; day 7; day 14), subcutaneously (at the base of the tail) with preparations as described in Table 4.

TABLE 4

| | Number of mice | Antigen (dose and | Other |
|---|---|---|---|
| | per group | volume per mouse) | treatment |
| Experiment N°1 | 5 | HPV16E7 protein | — |
| | | (10 μg in 100 μl) | |
| Experiment N°2 | 5 | HPV16E7 protein | NA fraction |
| | | (10 μg in 100 μl) | (25 μg) |
| Experiment N°3 | 5 | HPV16E7 protein | NA-B2 |
| | | (10 μg in 100 μl) | fraction |
| | | | (25 μg) |

Animals were then sacrificed 7 days after the last injection and their splenocytes were used to determine the frequency of R9F specific CD8+ T cells secreting IFN-γ upon re-stimulation.

The ELISpot plate was coated with Rat anti-mouse IFN-γ monoclonal antibody (100 μl/well; BD Pharmingen, ref: 551216) diluted at 2.5 μg/ml in sterile DPBS. The plate was then covered and incubated either overnight at room temperature or 4 h at 37° C. or 24 h at 4° C. 5 washes with sterile PBS (200 μl/well) were then performed. The plate was then blocked for 1 h at 37° C. with 200 μl/well of complete medium.

To prepare the lymphocytes for the experiment, 5 ml of Complete Medium (RPMI; FBS 10%; 40 μg/ml Gentamycin; 2 mM Glutamine; 5×10⁻⁵M b-mercaptoethanol) was put per well in 6-wells plate. The spleens from the same group of mice were pooled in a cell strainer (BD Bioscience; Ref. 352360) in a well of 6-well culture plates. The spleens were crushed with a syringe piston and the cell strainer was discarded. The splenocytes were collected with 5 ml of Complete Medium and then transferred in a 15 ml falcon tube on ice. Centrifugation during 3 min at 400×g and at room temperature (22° C.) was then performed. Cells were re-suspended in 8 ml of Complete Medium at room temperature. 8 ml of lymphocytes or splenocytes suspension were laid over 4 ml of Lympholyte®-M separation cell media (TEBU BIO, ref: CL5031). Centrifugation during 20 min at 1500×g at room temperature (22° C.) was then performed. The lymphocytes were collected, ringed and rinsed three times with 10 ml of RPMI minimum medium. A centrifugation was performed (during 3 min at 400×g) between each of the rinse step and supernatant was discarded. The lymphocytes were then re-suspended in 2 ml of RBC lysis buffer (BD Pharmingen; Ref. 555899). Each tube was gently vortexed immediately after adding the lysis solution and then incubated at room temperature for 15 minutes. Centrifugation during 3 min at 400×g was then performed and the supernatant was discarded. Cells were washed with 10 ml of Complete Medium and then centrifuged during 3 min at 400×g. The supernatant was discarded. After re-suspension of the cells in 6 ml of Complete Medium (depending on the size of the pellet), the cells were numerated on Malassez cells and the cell concentration was adjusted at 1×10⁷ cells per ml in Complete Medium.

The ELISpot assay itself is performed as follow: 100 μl of Complete Medium were added per well with or without 2-4 μg/ml of peptide of interest (i.e. HPV16E7 peptidic antigen). 100 μl of cell suspension were added. After incubation at 37° C. in 5% CO₂ for 20 h, two washing steps with H₂O wash buffer (PBS, 1% PBS) followed by five washing steps in PBS wash buffer were performed (tap dry). Biotinylated rat anti-mouse IFN-γ monoclonal antibody (BD Pharmingen, ref: 554410) was diluted at 4 μg/ml in antibody mix buffer and distributed 100 μl/well. The plate was incubated 2 h at room temperature in darkness. Five washing steps in PBS wash buffer (PBS, 0.05% Tween 20) were performed (tap dry). Streptavidin-Phosphatase alkaline was then diluted ( 1/1000) in antibody mix buffer. 100 μl/well were added and incubated 1 h at room temperature in darkness. Five washing steps in PBS wash buffer followed by two washing steps with PBS were then performed (tap dry). 100 μl/well of BCIP/NBT (SIGMA; Ref. B5655) were then added and incubated at room temperature until development of blue spots (for 2 min maximum). After thoroughly rinsing with water (tap dry), the analysis of ELISpot plates was performed with an ELISpot reader. Visual quality control (comparison of scans and plates) was performed on each well to ensure that the counts given by computer match the reality of the picture (removal of potential artefacts). Raw data were transformed into histogram graph. Results are expressed as number of spot forming units (sfu) per 1×10⁶ lymphocytes (mean) for each triplicate. A cut-off has been determined using non re-stimulated wells using the formula: [mean(non re-stimulated wells)]+[2×SD(non re-stimulated wells)]. The level of non specific background is revealed by re-stimulation with the irrelevant I8L peptide (HPV16E1).

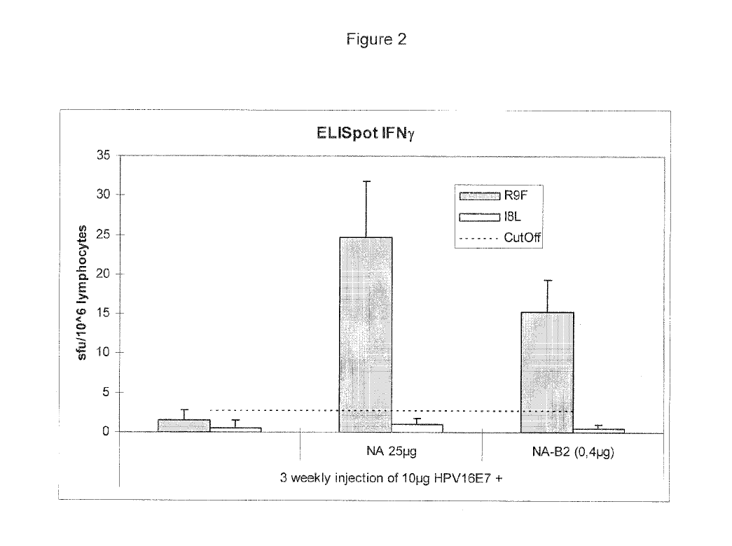

Results as depicted in FIG. 2 show that:
(1) There are no R9F specific (HPV16E7 protein) T cells secreting IFN-γ upon injection with HPV16E7;
(2) The level of R9F specific cells secreting IFN-γ following the addition of NA fraction (25 μg) or NA-B2 fraction (0.4 μg) to HPV16E7 protein is significant. NA fraction and NA-B2 fraction are endowed with an adjuvant capacity that specifically results in an increased frequency of circulating CD8+ T cells able to secrete the Th1 Cytokine IFN-γ upon re-stimulation.

### Example 5

### Use of NA Fraction and MUC-1 Antigen for the Preparation of a Pharmaceutical Composition Intended for the Treatment of Cancers

Denomination and brief description of each vector construction (see Table 5)

TABLE 5

| | | Batch |
|---|---|---|
| Virus | | concentration |
| Denomination | Transgene | (pfu/ml) |
| MVAN33 | — | 7.9 10⁸ pfu/ml |
| MVA9931 | MUC1-NL-2 | 8.2 10⁸ pfu/ml |

Animal model used are SPF healthy female B6D2 mice were as described in Example 3.

RenCa-MUC-1 tumor cells: RenCa is an experimental murine kidney cancer model (Chakrabarty A. et al. Anticancer Res. 1994; 14:373-378; Salup R. et al. Cancer Res 1986 46: 3358-3363). RenCa-MUC-1 cells were obtained after transfection of a plasmid expressing MUC-1 peptide. Such cells expressed the MUC1 antigen on their surface. RenCa-MUC-1 cells were cultured in DMEM containing 10% inactivated foetal calf serum, 2 mM L-glutamin, 0.04 g/l gentamycin and 0.6 mg/ml Hygromycin.

Immunization: For the immunotherapeutic experiments. B6D2 female mice were challenged subcutaneously in the right flank with 3·10⁵ RenCa-MUC 1 cells at day 1. Mice were treated three times, subcutaneously with the vehicle alone (Buffer), 5·10⁷ pfu of MVA-null (MVAN33), NA fraction (50 μg), Lipofectin® (50 μg; Invitrogen, Cat. No. 18292-011 or Cat. No. 18292-037), NA+Lipofectin® (50 μg+50 μg), 5·10⁷ pfu of MVA strain expressing MUC1 and hIL-2 (MVA9931) alone or in combination with NA fraction (50 μg) or NA+Lipofectin® (50 μg+50 μg) (13 mice per group) at day 4, day 11 and day 18. Mice were also treated three times intratumorally with NA+Lipofectin® (50 μg+50 μg) alone at day 4, day 11 and day 18. Injection scheme: MVA9931 was injected first; 1 h later NA fraction or NA+Lipofectin® was injected at same site. Survival of mice was monitored. Tumor volume was also monitored, twice a week using a caliper. Mice were euthanised for ethical reasons when their tumor size was superior to 25 mm of diameter.

Statistics: Kaplan-Meier survival curves were analyzed by the log-rank test using Stastistica 7.1 software (StatSoft, Inc.), and specific pairwise comparisons were made. A P<0.05 was considered to be statistically significant.

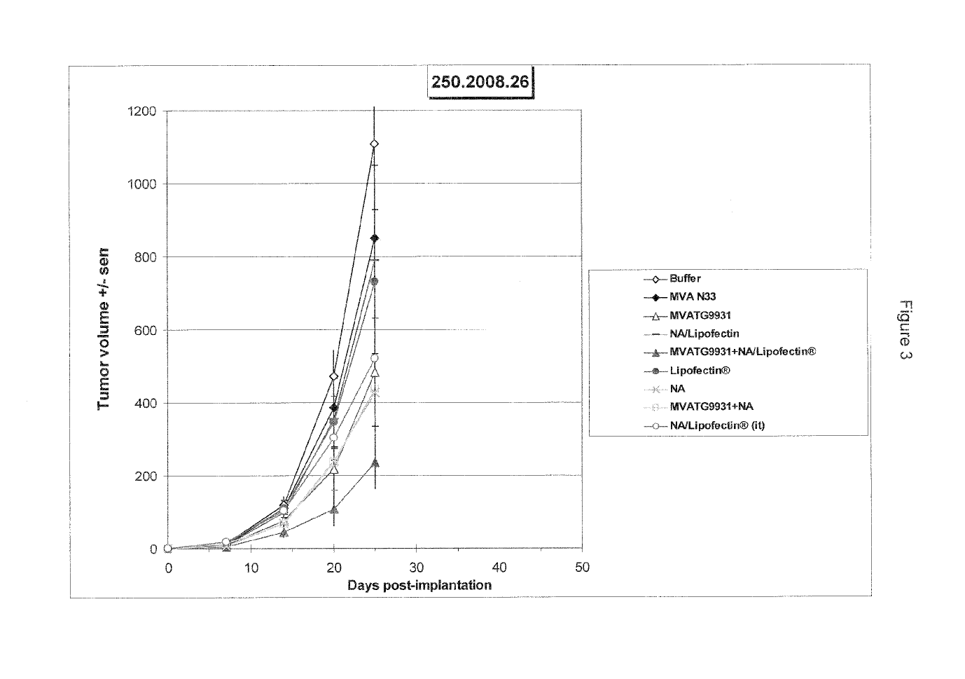

Results as depicted in FIG. 3 show that compared to the untreated control, MVATG9931 in combination with NA fraction (50 μg) or NA+Lipofectin® (50 μg+50 μg) had statistically significant effects on tumor growth day 20 (p: 0.007752) and day 25 (p: 0.023046).

### Example 6

### Induction of the Cytokines Gamma Interferon (IFN-γ), Interleukin 12 (IL-12) and Alpha Interferon (IFN-α) in Human Immature Monocyte-Derived Dendritic Cells (moDCs) Treated with NA-B2 Fraction, Lipofectin®, Ambisome®, NA-B2+Lipofectin® and/or NA-B2+Ambisome®

Cell culture: Elutriated human monocytes from healthy volunteers were obtained from the Etablissement Français du Sang-Alsace (EFS). Frozen cells were taken into culture at a concentration of 1×10⁶ cells/ml in RPMI (Gibco) supplemented with 10% inactivated Fetal Calf Serum, 40 μg/ml Gentamycine (Sigma), 2 mM L-Glutamine (Sigma), 1 mM Sodium Pyruvat (Sigma, S8636) and 1× Non Essential Amino Acids (MEM NEAA, GIBCO). To induce differentiation of elutriated monocytes to dendritic cells (moDCs), the cytokines GM-CSF (20 ng/ml) and IL-4 (10 ng/ml) (Peprotech) were added. Three days later, cells were counted, centrifuged and taken up in fresh supplemented medium at a density of 1×10⁶ cells/ml. Two×10⁶ cells were plated in 12 well plates (2 ml/well). After another 2 to 3 days, cells considered to be immature moDCs were infected and/or stimulated as indicated below.

Stimulation: NA-B2 fraction, Lipofectin® (Invitrogen, Cat. No. 18292-011 or Cat. No. 18292-037) and Ambisome® (Gilead Sciences) were added to the moDCs. After 16-20 h, cells were centrifuged, the supernatants were stored at −20° C. and analyzed by ELISA.

Detection of cytokines by Elisa: The amount of cytokine production was determined after 16-20 h stimulation using commercially available ELISA kits from Bender Med System (IFNγ, IL12(p70) and IFNα). The ELISA assays were performed according to the manufacturer's protocol. The concentration of cytokines was determined by standard curve obtained using known amounts of recombinant cytokines.

### Results:

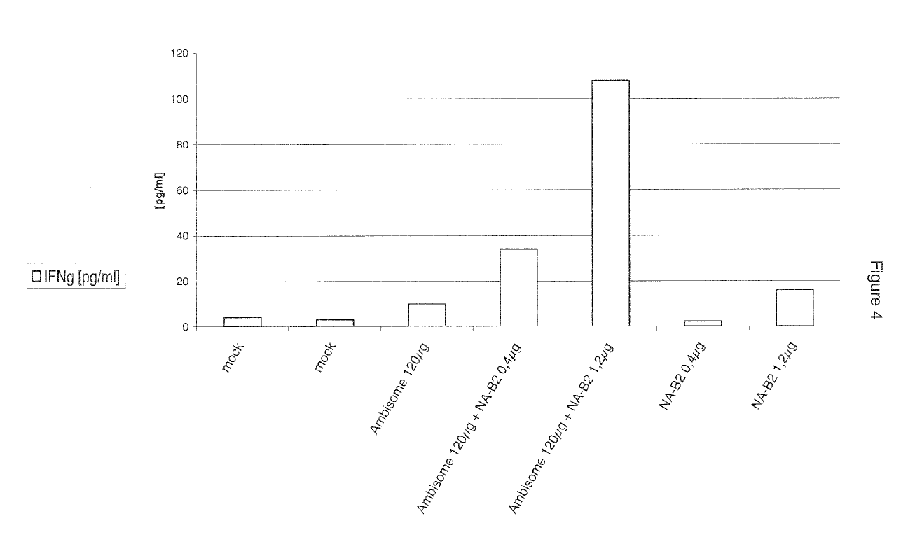

Gamma interferon (IFN-γ): As depicted in FIG. 4, gamma interferon expression was induced by the NA-B2 fraction alone (0.4 μg or 1.2 μg) as well as by Ambisome® alone (120 μg); but the gamma interferon expression level obtained by treatment of human immature moDCs with NA-B2 fraction 1.2 μg is higher than the gamma interferon expression level obtained by treatment of human immature moDCs with Ambisome® 120 μg. Moreover, added together, the NA-B2 fraction and Ambisome® (0.4 μg+120 μg or 1.2 μg+120 μg) increase the gamma interferon expression in a synergistic manner.

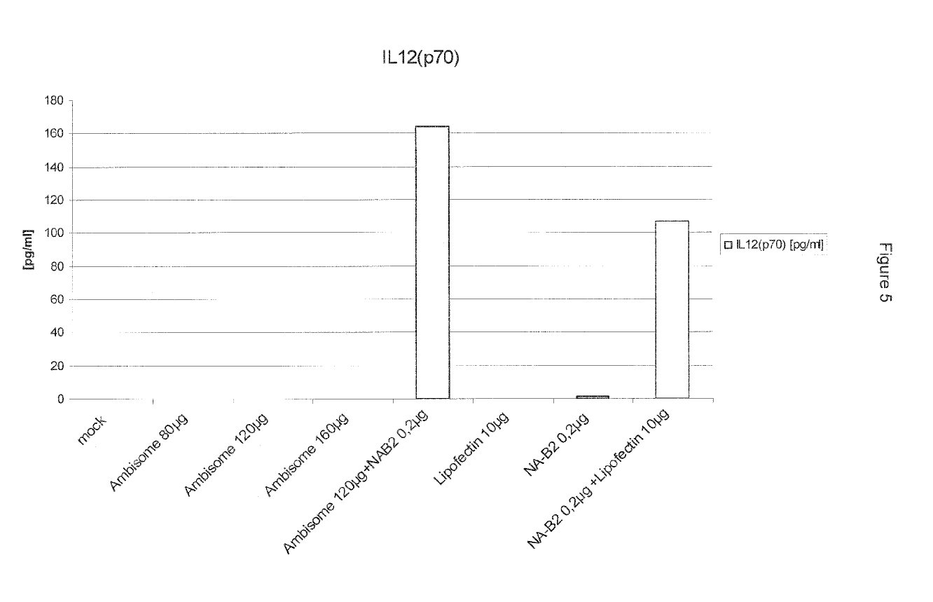

Interleukin 12 (IL-12): As depicted in FIG. 5, human immature moDCs treated with NA-B2 fraction 0.2 μg slightly produce IL-12 whereas human immature moDCs treated with Lipofectin® 10 μg or with Ambisome® 80 μg, 120 μg or 160 μg, do not secrete IL-12. The combination NA-B2+Lipofectin® (0.2 μg+10 μg) and the combination NA-B2+Ambisome® (0.2 μg+120 μg) added to human immature moDCs clearly stimulate the secretion of IL-12 (synergic effect).

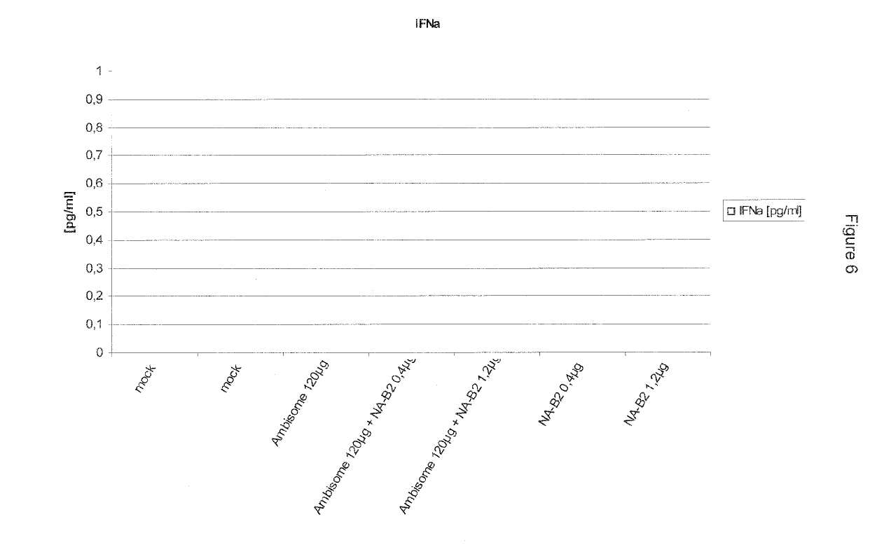

Alpha interferon (IFN-α): As depicted in FIG. 6, the NA-B2 fraction alone (0.4 μg or 1.2 μg). Ambisome® alone (120 μg) as well as the combination NA-B2+Ambisome® (0.4 μg+120 μg or 1.2 μg+120 μg) do not induce alpha interferon (IFN-α).

All documents (e.g. patents, patent applications, publications cited in the above specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for orienting the immune response of a mammal toward a Th1 type response comprising administering to the mammal a pharmaceutical composition comprising a Saccharomyces cerevisiae mitochondrial nucleic acids fraction and an antigen, wherein the Saccharomyces cerevisiae mitochondrial nucleic acids fraction is prepared by a method comprising:
a) culturing Saccharomyces cerevisiae in a culture medium allowing growth, and centrifuging said culture to obtain a Saccharomyces cerevisiae pellet;
b) suspending the pellet obtained in step a) in buffer and agitating the resulting suspension;
c) centrifuging the suspension obtained in step b) to obtain a supernatant;
d) ultracentrifuging the supernatant obtained in step c) to obtain a pellet;
e) extracting nucleic acids from the pellet obtained in step d), and obtaining a supernatant comprising the nucleic acids fraction; and
f) recovering the nucleic acids fraction from the supernatant obtained in step e).

2. The method according to claim 1, wherein the nucleic acids are ribonucleic acids (RNA).

3. The method according to claim 1, wherein the antigen is a tumor associated antigen (TAA), an antigen specific to an infectious organism, or an antigen specific to an allergen.

4. The method according to claim 1, wherein the antigen is a peptide, a nucleic acid, a lipid, a lipopeptide, or a saccharide.

5. The method according to claim 3, wherein the antigen is a TAA, and the TAA is MUC-1.

6. The method according to claim 3, wherein the antigen is an antigen specific to Human Papilloma Virus (HPV).

7. The method according to claim 1, wherein the antigen is comprised in a vector.

8. The method according to claim 7, wherein the vector is a plasmid or a viral vector.

9. The method according to claim 8, wherein the vector is a viral vector obtained from an adenovirus, an adenovirus-associated virus, a retrovirus, a herpesvirus, an alphavirus or a foamy virus, or a derivative thereof.

10. The method according to claim 7, wherein the vector further comprises the elements necessary for the expression of the antigen when the antigen is a nucleic acid.

11. The method according to claim 1, wherein the pharmaceutical composition further comprises one or more agent that improves the transfectional efficiency and/or the stability of the Saccharomyces cerevisiae mitochondrial nucleic acids fraction and/or of the antigen.

12-14. (canceled)

15. An composition comprising:
(i) a Saccharomyces cerevisiae mitochondrial nucleic acids fraction, wherein the Saccharomyces cerevisiae mitochondrial nucleic acids fraction is prepared by a method comprising:
a) culturing Saccharomyces cerevisiae in a culture medium allowing growth, and centrifuging said culture to obtain a Saccharomyces cerevisiae pellet;
b) suspending the pellet obtained in step a) in buffer and agitating the resulting suspension;
c) centrifuging the suspension obtained in step b) to obtain a supernatant;
d) ultracentrifuging the supernatant obtained in step c) to obtain a pellet;
e) extracting nucleic acids from the pellet obtained in step d), and obtaining a supernatant comprising the nucleic acids fraction; and
f) recovering the nucleic acids fraction from the supernatant obtained in step e); and
(ii) liposomal amphotericin-B.

16. A composition comprising:
(i) a Saccharomyces cerevisiae mitochondrial nucleic acids fraction, wherein the Saccharomyces cerevisiae mitochondrial nucleic acids fraction is prepared by a method comprising:
a) culturing Saccharomyces cerevisiae in a culture medium allowing growth, and centrifuging said culture to obtain a Saccharomyces cerevisiae pellet;
b) suspending the pellet obtained in step a) in buffer and agitating the resulting suspension;
c) centrifuging the suspension obtained in step b) to obtain a supernatant;
d) ultracentrifuging the supernatant obtained in step c) to obtain a pellet;
e) extracting nucleic acids from the pellet obtained in step d), and obtaining a supernatant comprising the nucleic acids fraction; and
f) recovering the nucleic acids fraction from the supernatant obtained in step e);
(ii) liposomal amphotericin-B; and
(iii) an antigen.

17. A composition comprising:
(i) a Saccharomyces cerevisiae mitochondrial nucleic acids fraction prepared by a method comprising:
a) culturing Saccharomyces cerevisiae in a culture medium allowing growth, and centrifuging said culture to obtain a Saccharomyces cerevisiae pellet;
b) suspending the pellet obtained in step a) in buffer and agitating the resulting suspension;
c) centrifuging the suspension obtained in step b) to obtain a supernatant;
d) ultracentrifuging the supernatant obtained in step c) to obtain a pellet;
e) extracting nucleic acids from the pellet obtained in step d), and obtaining a supernatant comprising the nucleic acids fraction; and
f) recovering the nucleic acids fraction from the supernatant obtained in step e); and
(ii) dioleoyl phosphatidylethanolamine (DOPE) and N-[1-(2,3-dioleyloxy)propyl]-N,N,N-trimethylammonium chloride (DOTMA).

18. A composition comprising:
(i) a Saccharomyces cerevisiae mitochondrial nucleic acids fraction prepared by a method comprising the following steps:
a) culturing Saccharomyces cerevisiae in a culture medium allowing growth, and centrifuging said culture to obtain a Saccharomyces cerevisiae pellet;
b) suspending the pellet obtained in step a) in buffer and agitating the resulting suspension;
c) centrifuging the suspension obtained in step b) to obtain a supernatant;
d) ultracentrifuging the supernatant obtained in step c) to obtain a pellet;
e) extracting nucleic acids from the pellet obtained in step d), and obtaining a supernatant comprising the nucleic acids fraction; and
f) recovering the nucleic acids fraction from the supernatant obtained in step e);
(ii) dioleoyl phosphatidylethanolamine (DOPE) and N-[1-(2,3-dioleyloxy)propyl]-N,N,N-trimethylammonium chloride (DOTMA); and
(iii) an antigen.

19. A kit comprising a container containing at least one Saccharomyces cerevisiae mitochondrial nucleic acids fraction, a container containing at least one antigen, and instructions for administrating said nucleic acids fraction and antigen to a mammal.

20. A kit comprising a container containing at least one Saccharomyces cerevisiae mitochondrial nucleic acids fraction, a container containing at least one antigen, a container containing at least one agent that improves the transfectional efficiency and/or the stability of the Saccharomyces cerevisiae mitochondrial nucleic acids fraction and/or the antigen, and instructions for administering said nucleic acids fraction and antigen.

21. A method for treating cancer, an infectious disease, allergy, and/or an autoimmune disorder, comprising administering to a mammal the pharmaceutical composition of claim 1.

22. The method according to claim 6, wherein the antigen specific to the HPV is an antigen specific to at least one of HPV-16 and HPV-18.

23. The method according to claim 22, wherein the antigen specific to at least one of HPV-16 and HPV-18 is an E6 early coding region of HPV-16 and/or HPV-18, an E7 early coding region of HPV-16 and/or HPV-18, or a combination thereof.

24. The method according to claim 8, wherein the viral vector is obtained from a poxvirus.

25. The method according to claim 24, wherein the poxvirus is a vaccinia virus.

26. The method according to claim 25, wherein the vaccinia virus is a modified vaccinia virus Ankara (MVA).

27. The method according to claim 11, wherein the one or more agent is a lipid, a liposome, a submicron oil-in-water emulsion, a microparticle, an ISCOM, or a polymer.

28. The method according to claim 27, wherein the liposome is a cationic liposome.

29. The method according to claim 28, wherein the cationic liposome is one of or a combination of dioleoyl phosphatidylethanolamine (DOPE), N-[1-(2,3-dioleyloxy)propyl]-N,N,N-trimethylammonium chloride (DOTMA) and liposomal amphotericin-B.

30. The method according to claim 29, wherein the cationic liposome is dioleoyl phosphatidylethanolamine (DOPE) and N-[1-(2,3-dioleyloxy)propyl]-N,N,N-trimethylammonium chloride (DOTMA).
